(19) United States
(12) Patent Application Publication
(10) Pub. No.: US 2024/0209052 A1
(43) Pub. Date: Jun. 27, 2024

(54) ACTIVATABLE POLYPEPTIDE SEQUENCES FOR PREPARING CYCLIZED POLYPEPTIDES

(71) Applicant: National Tsing Hua University, Hsinchu (TW)

(72) Inventor: Shih-Che Sue, Hsinchu (TW)

(73) Assignee: National Tsing Hua University, Hsinchu, TW (TW)

(21) Appl. No.: 18/395,769

(22) Filed: Dec. 26, 2023

Related U.S. Application Data
(60) Provisional application No. 63/477,225, filed on Dec. 27, 2022.

(51) Int. Cl. C07K 14/55; C07K 14/00; C07K 14/54

(52) U.S. Cl. CPC C07K 14/55; C07K 14/001; C07K 14/5412; C07K 14/5443

(57) ABSTRACT

Protein cyclization is a method for making proteins more stable. The method requires a properly designed linker to connect the original N-terminus and C-terminus of a protein sequence. A new strategy is used for creating flexibility in the linker design. The new strategy includes an activatable polypeptide arrangement to produce cyclized proteins, characterized in that the strategy can be used to produce cyclized interleukins. The strategy cyclizes the interleukins via introducing a linker into the interleukin and using an intein-mediated protein splicing. The linker sequence is not related to the protein splicing reaction, thus providing flexibility in the introduction of linker. The prepared cyclized interleukins contain high structural stability, native interleukin structure and long-lasting activity. The cyclized interleukins have the potential to replace native interleukins in bio-industrial applications or to modulate the functions of interleukins.

This application claims the benefit of U.S. Provisional Patent Application No. 63/477,225 filed on Dec. 27, 2022, which is incorporated by reference herein in its entirety.

This application contains a Sequence Listing in a computer readable form, the file name is 4104-NTHU-SEQLIST, created on Dec. 22, 2023, the size is 38 KB, which is incorporated herein by reference.

### FIELD OF THE INVENTION

The present invention relates to anew strategy to produce cyclic proteins characterized in that it is feasible to use the strategy to prepare cyclic interleukins to execute the functions of interleukins and replace interleukins in bio-industrial applications or modulate the functions of interleukins.

### DESCRIPTION OF PRIOR ART

Cyclic protein owns the structural benefit of rigidifying and stabilizing protein. To achieve the purpose of protein cyclization, the common strategy is to condense the N- and C-terminal ends of a polypeptide. In most cases, a suitable linker is required to be integrated into the protein sequence and connect the original N-terminus and C-terminus of the protein sequence. To prepare an effective cyclic protein, the sequence and length of the linker should be properly designed and the linker is important in maintaining protein folding and function.

The idea has been extensively used in enhancing the protein structural stability in many cases. There are some developed methods used in preparing cyclic peptide and protein, such as native chemical ligation (NCL), expressed protein ligation (EPL) and intein-based protein splicing. The methods provided different strategies in linking the native N- and C-terminal ends of a polypeptide chain. Among the methods, intein-based protein splicing becomes a very promising method for producing a cyclic polypeptide. Intein-based protein splicing is a process in which a protein undergoes an intramolecular rearrangement where an internal sequence, named “intein”, is excised by itself and the lateral sequences, respectively named N- and C-terminal “exteins”, is joined. The method has been extended to use a “split intein” to mediate the cyclization process. The native intein sequence is divided into two fragments, named N-intein and C-intein, respectively and the two fragments own the ability to assemble into an entire intein fold to execute the protein splicing function. Split intein-based protein cyclization is in the context of a fusion protein precursor of the primary sequence, “C-intein—target sequence—N-intein”. The design results in head-to-tail cyclization of the target sequence (FIG. 1).

### BRIEF SUMMARY OF THE INVENTION

The present invention provides methods to biologically produce a cyclic protein through a single polypeptide sequence. The single polypeptide sequence can be used in protein expression in any biological expression system (such as E. coli, yeast and any different expression cells). The single polypeptide sequence comprises three portions: split intein sequence (containing two respective fragments of N-intein and C-intein), linker and split target protein sequence. After protein expressed, the target protein sequence will be automatically cleaved from the primary sequence and cyclized by the help of split intein. The cyclization reaction occurs robotically and no need for incorporating any chemical reaction or ATP/coenzyme molecules. The linker sequence can be arbitrarily introduced.

Therefore, the present invention provides a new arrangement for the single polypeptide sequence.

The classic arrangement adopts the sequence of “C-intein—linker1—target sequence—(linker2)—N-intein”. The linker sequence is required to be well designed to ensure the efficiency of protein splicing reaction.

The present invention provides a new way to prepare the cyclized protein. The present invention rearranges the primary sequence. The target sequence is firstly separated into two sequences: “N-target” and “C-target” fragments, wherein the split site is rationally selected. A linker is introduced between the original N- and C-terminal ends of the target sequence. The new arrangement of the primary sequence is “C-intein—C-target—linker—N-target—N-intein” (FIG. 2B). Since the linker sequence is not related to protein splicing reaction, there is no limitation in introducing any sequence as the linker.

The new invention provides flexibility in customizing the linker used in cyclization. Linkers herein can be adjusted according to the need of modulating protein/polypeptide function, including targeting various receptors, regulating activation and assisting purification.

The present invention has the new invention and proves the feasibility in the usage of producing cyclized interleukins. Interleukin family has important functions in regulating immune responses and immune cell migration and proliferation. The features extend the therapeutic usage of interleukins.

The members of interleukin own general features of protein instability. The design of cyclic interleukins creating stable and structurally folded constructs promotes the applications of interleukins in all aspects.

With proper design, the method of the new invention can be used to replace the method of the classic method in producing cyclized interleukins. The new invention provides greater flexibility in introducing linker.

The same strategy will be extended to other protein systems with the same structure property, such as flexible N- and C-termini in close proximity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the split intein-based protein splicing for producing cyclized proteins. It contains three elements of C-intein, target protein and N-intein. The ligation junction must contain a residue providing the side-chain nucleophile (—SH and —OH). The residue is required to be placed in the N-terminal end of the target sequence in which the side-chain nucleophile would be involved in the following transesterification reaction.

FIG. 2 shows the two designs for producing cyclic protein. FIG. 2A is the scheme of the classic method for producing cyclic protein. The arrangement of the protein is “C-intein—linker 1—target sequence—linker 2—N-intein”. FIG. 2B is the scheme of the present invention for producing cyclized protein. The arrangement of the protein is “C-intein—C-target-linker—N-target—N-intein”.

FIG. 3 shows the purification and characterization of the cyclized IL-2. FIG. 3A is the SDS-PAGE analysis confirming the expression and purification of cyclic IL-2 of the present invention. The expressed protein polypeptide is actively self-cleaved, and the N-intein and C-intein are excised from the cyclic IL-2 sequence. I⁺: IPTG induction; P: pellet of cell lysis; S: supernatant of cell lysis; RS: supernatant after refolding from pellet; RP: precipitation after refolding from pellet; 1-3: fractions from fast performance liquid chromatography (FPLC) gel filtration purification. The samples prepared with adding β-mercaptoethanol (0-ME) is denoted by “+”. FIG. 3B shows the fractions of 1, 2 and 3 in the FPLC gel filtration profile of cyclized IL-2 C5A. The protein in fraction 3 represents the purified monomeric cyclic IL-2 (arrow). FIG. 3C shows the purity of the purified cyclized IL-2 C5A as proved by mass result. FIG. 3D shows the fraction in the FPLC gel filtration profile of cyclized IL-2 G5A. The fraction represents the purified monomeric cyclic IL-2 (arrow). FIG. 3E shows the purity of the purified cyclized IL-2 G5A as proved by mass result.

FIG. 4 shows the NMR heteronuclear single quantum coherence (HSQC) spectra of cyclized IL-2 C5A and G5A. FIG. 4A shows that the spectral similarity indicates the structural similarity between the two cyclized IL-2s. The nuclear magnetic resonance (NMR) condition is with the buffer of 20 mM sodium phosphate, pH 7.4, 150 mM NaCl and temperature at 298K. FIG. 4B shows that the HSQC resonance distribution of native IL-2 is simulated based on the deposited data (Biological Magnetic Resonance Data Bank, BMRB code 28104). Comparing to the chemical shifts of native IL-2, the cyclized IL-2 adopts a similar resonance distribution.

FIG. 5 shows the strip plots of NMR 3D HNCA experiment to report the backbone connection of IL-2 G5A. FIG. 5A shows that the sequential assignment represents the residues between L56 and E60, where the split junction is introduced between residues Q57 and C58. FIG. 5B shows that the sequential assignment represents the residues between T133 and Al which include the GSGSG linker to connect the N- and C-terminal ends. The NMR condition is with buffer of 20 mM sodium phosphate, pH 6, 150 mM NaCl and temperature at 303K. The successful sequential assignment reveals the backbone connection, therefore proving the success of cyclization using the present invention. FIG. 5C shows the backbone secondary structure prediction based on the differences between the measured chemical shifts of C_{α} and C_{β} and the corresponding chemical shifts in a random coil. The differences are noted as ΔC_{α} and ΔC_{β}. In a α-helix structure, the parameter of ΔC_{α}-ΔC_{β} (ppm) is a positive value and in a β-sheet structure, the parameter is a positive value.

FIG. 6 shows the correlation between the experimental chemical shifts (¹H and ¹⁵N) of IL-2 G5A and reported chemical shifts (¹H and ¹⁵N) of native IL-2 (BMRB code 28104). The linear correlations in ¹H and ¹⁵N chemical shifts indicate the same structure between IL-2 G5A and native IL-2. Only few residues that distribute near the linker region have different chemical shifts.

FIG. 7 shows the structural stability of the cyclized IL-2 G5A proved by the consistency of HSQC spectra of the cyclized IL-2 G5A acquired after different storage periods at 4° C.

FIG. 8 shows the thermal denaturation tendency, monitored by circular dichroism (CD) spectroscopy. FIG. 8A shows the structural stability of cyclized IL-2, monitored by circular dichroism (CD) spectra at different temperatures where IL-2 G5A is selected as a representative case. FIG. 8B shows the profile of thermal denaturation of cyclized IL-2 G5A, that is monitored by CD spectra at one single wavelength of 208 nm. The measured melting temperature is higher than 70° C.

FIG. 9 shows the cell proliferation activity of cyclized IL-2 using a mouse CTLL-2 cell model where IL-2 G5A is selected as a representative case.

FIG. 10 shows the structural stability of cyclized IL-15s and native IL-15, monitored by circular dichroism (CD) spectra at different temperatures.

FIG. 11 shows the HSQC spectrum of cyclized IL-15 G4A, IL-15 G5A, IL-15 G6A. The dispersive resonances indicate a well-folded structure. The NMR condition is with a buffer of 25 mM Tris, pH 6.5, 120 mM NaCl and temperature at 298K.

FIG. 12 shows the cell proliferation activity of cyclized IL-15 G6A using a mouse CTLL-2 cell model where IL-15 G6A is selected as a representative case.

FIG. 13 shows the HSQC spectra of cyclized IL-6 G5A, IL-6 G5B, and the native IL-6. FIG. 13A shows the HSQC spectra of cyclized IL-6 G5A. FIG. 13B shows the HSQC spectra of cyclized IL-6 G5B. FIG. 13C shows the HSQC spectra of native IL-6. The NMR condition is with buffer of 20 mM sodium phosphate, pH 6.0, 150 mM NaCl and temperature at 298K.

FIG. 14 shows the structural stability and melting temperature of IL-6. FIG. 14A shows the structural stability of cyclized IL-6 G5B, monitored by circular dichroism (CD) spectra at different temperatures. The thermal denaturation is monitored by CD spectra at one single wavelength of 208 nm. The measured melting temperature is 81° C. FIG. 14B shows the structural stability of native IL-6, monitored by circular dichroism (CD) spectra at different temperatures. The thermal denaturation is monitored by CD spectra at one single wavelength of 208 nm. The measured melting temperature is 68° C.

### DETAILED DESCRIPTION OF THE INVENTION

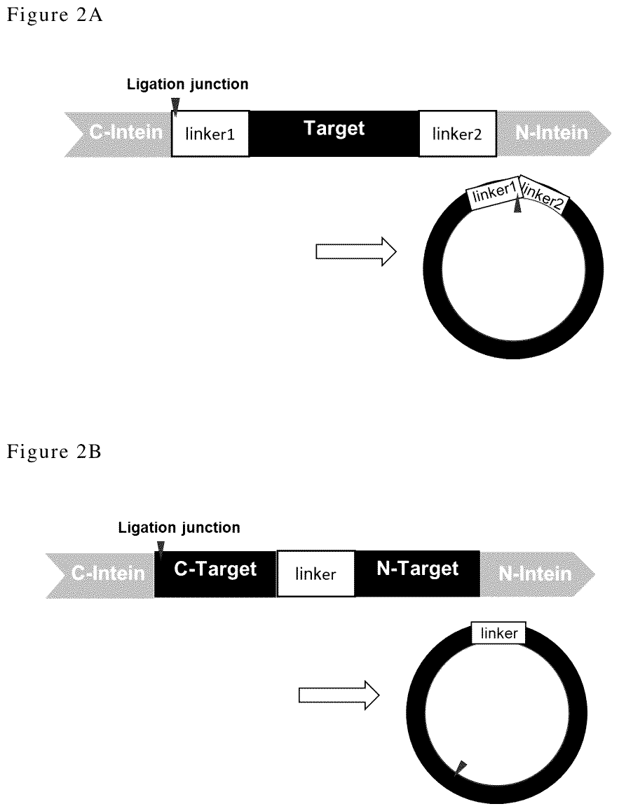

The classic arrangement of the cyclic protein is that C-intein specifically binds to the first linker, wherein the other end of the first linker is fused to the N-terminal end of target protein sequence. The linker could be optimized depending on the requirement of linker. If required, the C-terminal end of the target protein sequence binds to the second linker. The second linker is further fused to the N-terminal end of N-intein. Otherwise, the target protein sequence can be directly fused to the N-terminal end of N-intein. Therefore, the arrangement adopts the sequence of “C-intein—linker 1—target sequence—(linker 2)—N-intein” (FIG. 2A). The linker sequence is required to be well designed to ensure the efficiency of protein splicing reaction.

The present invention provides a new way to prepare the cyclized protein. The present invention rearranges the primary sequence differently than the classic arrangement. The target sequence is firstly separated into two sequences, wherein the split site in the target sequence is rationally selected. The sequence before the split site is the “N-target” fragment and the other sequence after the split site is the “C-target” fragment. A linker is introduced between the original N- and C-terminal ends of the target sequence. The new invention with the arrangement of the primary sequence is “C-intein—C-target-linker—N-target—N-intein” (FIG. 2B). Since the linker sequence is not related to protein splicing reaction, there is no limitation in introducing any sequence as the linker.

The present invention proves the feasibility of the usage of producing cyclized interleukins. Interleukin family has important functions in regulating immune responses and immune cell migration and proliferation. The features extend the therapeutic usage of interleukins. The important applicants include acting ingredients in cell culture medium to enhance cell growth, supplementary proteins for cell therapy, and protein drugs for cancer therapy. However, the members of interleukin own general features of protein instability. The circulation time in vivo could be as short as several minutes. Therefore, the design of cyclic interleukins creating stable and structurally folded constructs promotes the applications of interleukins in all aspects.

With proper design, the method of the present invention can be used to replace the method of classic method in producing cyclized proteins. The present invention provides great flexibility in introducing a linker.

The present invention provides a method to produce cyclized proteins in which an arbitrary sequence can be set as the linker, meaning any linker sequence and any linker length can be used to connect the N- and C-terminal ends of an native protein sequence to create the new cyclic proteins. The present invention demonstrates the usage in the members of interleukin family and proves the feasibility. The same strategy will be extended to different protein systems with the same structure property, such as flexible N- and C-termini in close proximity.

As used herein, “a,” “an,” “the,” “at least one,” and “one or more” are used interchangeably.

As used herein, the term “a” or “an” are employed to describe elements and components of the present invention. This is done merely for convenience and to give a general sense of the present invention. This description should be read to include one or at least one and the singular also includes the plural unless it is obvious that it is meant otherwise.

The term “or” as used herein may mean “and/or.”

As used herein, the terms “polypeptide”, “protein” and “peptide” are used interchangeably and mean a polymer of amino acids not limited to any particular length.

In the present invention, the residue “C” means cysteine, the residue “F” means phenylalanine, the residue “N” means asparagine, the residue “G” means glycine, and the residue “S” means serine.

The present invention uses the carboxy-terminal portion of a split intein (C-intein) and the amino-terminal portion of a split intein (N-intein) to mediate the cyclization of interleukin.

The classic arrangement of the present invention provides an activatable polypeptide comprising, in the N-terminal to C-terminal direction: (1) a C-intein; (2) a first linker; (3) an interleukin; and (4) a N-intein, wherein the N-terminus of the first linker has an amino acid with a side-chain nucleophile.

The classic arrangement of the present invention also provides a method for producing an activatable polypeptide comprising: preparing a sequence arrangement of peptides to form the activatable polypeptide, wherein the sequence arrangement of peptides comprises, in the N-terminal to C-terminal direction: (1) a C-intein; (2) a first linker; (3) an interleukin; and (4) a N-intein, wherein the N-terminus of the first linker has an amino acid with a side-chain nucleophile.

In the classic arrangement of the present invention, the N-terminus of the first linker (linker 1) contains a ligation junction that provides a residue containing side-chain nucleophile (—SH and —OH). Thus, in the most situations, cysteine (Cys), serine (Ser) or threonine (Thr) is chosen to be the residue in the ligation junction. In a preferred embodiment, the amino acid with the side-chain nucleophile is cysteine.

In another embodiment, the activatable polypeptide further comprises a second linker which is placed between the interleukin and the N-intein.

In one embodiment, the sequences of the C-intein, the first linker, the interleukin, the second linker and the N-intein are the amino acid sequence.

In one embodiment, the N-intein and the C-intein are derived from the sequence of Npu DnaE intein.

In one embodiment, the C-intein comprises the peptide sequence of SEQ ID NO: 1, and the N-intein comprises the peptide sequence of SEQ ID NO: 2.

In one embodiment, the interleukin is interleukin-2, and the sequence of the interleukin-2 comprises the peptide sequence of SEQ ID NO: 3 or 4. In some aspects, the C-intein (SEQ ID NO: 1), the first linker (CFN), the interleukin (SEQ ID NO: 3), the second linker (GS) and the N-intein (SEQ ID NO: 2) are in order for forming the sequence of SEQ ID NO: 7 of IL-2 (IL-2 C5A). In addition, the C-intein (SEQ ID NO: 1), the first linker (CFN), the interleukin (SEQ ID NO: 4), the second linker (GS) and the N-intein (SEQ ID NO: 2) are in order for forming the sequence of SEQ ID NO: 8 of IL-2 (IL-2 C5B).

In one embodiment, the peptide sequence of the first linker comprises CFN, wherein the residue “C” is placed in the ligation junction and the residue “F” enhances the ligation activity. In another embodiment, the peptide sequence of the second linker comprises GS. There is no limitation in choosing residue types in the sequence of the second linker.

Therefore, the above activatable polypeptides using the classic method comprises the sequence arrangement of “C-intein—CFN—interleukin—GS—N-intein”, wherein the C terminus of the C-intein connects to the N terminal Cys residue of the first linker, the C terminus of the first linker connects to the N terminus of the interleukin, the C terminus of the interleukin connects to the N terminus of the second linker, and the C terminus of the second linker connects to the N terminus of the N-intein. In the classic method, the function of the ligation junction will connect the first linker and the second linker after the split inteins (C-intein and N-intein) are automatically excised.

The above activatable polypeptides (IL-2 C5A (SEQ ID NO: 7) or IL-2 C5B (SEQ ID NO: 8)) undergoes the protein splicing to form a cyclized interleukin having the first linker, the interleukin and the second linker. The cleaving is automatically from the protein self-splicing. When comparing to native IL-2, the cyclized IL-2 of the present invention has an additional linker of “GSCFN”. In one embodiment, the sequence of the cyclized interleukin comprises the peptide sequence of SEQ ID NO: 9 or 10.

The experiments of the present invention demonstrate that the sequence arrangement of the classic method is able to produce cyclized IL-2. The cyclized IL-2 had better stability, and similar functional activity in stimulating cell proliferation when comparing to the native interleukin. They can be used to replace the usage of native interleukin.

Based on the classic arrangement, the present invention further provides a cyclized polypeptide, which is in a circular form comprising a first linker, an interleukin and a second linker, wherein the C-terminus of the first linker is linked with the N-terminus of the interleukin, the C-terminus of the interleukin is linked with the N-terminus of the second linker, the C-terminus of the second linker is linked with the N-terminus of the first linker, and the N-terminus of the first linker has an amino acid with a side-chain nucleophile.

The present invention also provides a method for producing a cyclized polypeptide, comprising: (a) providing an activatable polypeptide comprising, in the N-terminal to C-terminal direction: (1) a C-intein; (2) a first linker; (3) an interleukin; (4) a second linker; and (5) a N-intein, wherein the N-terminus of the first linker has an amino acid with a side-chain nucleophile; and (b) making the activatable polypeptide to be subjected to an intein-mediated protein splicing for allowing splicing of the C-intein and the N-intein and linking of the N-terminus of the first linker to the C-terminus of the second linker to form the cyclized polypeptide.

In one embodiment, the peptide sequence of the first linker comprises CFN, and the peptide sequence of the second linker comprises GS.

In another embodiment, the interleukin comprises interleukin-2. In a preferred embodiment, the interleukin-2 comprises the peptide sequence of SEQ ID NO: 3, or 4.

In one embodiment, the sequence of the cyclized polypeptide comprises the peptide sequence of SEQ ID NO: 9 or 10.

In addition, the present invention also provides a new strategy to prepare the activatable cyclized protein. The present invention rearranges the primary sequence which is different than the classic arrangement. The target sequence is firstly separated into two sequences, wherein a split site is rationally selected in the target sequence. The sequence before the split site is the “N-target” fragment and the other sequence after the split site is the “C-target” fragment. A linker is introduced between the original N- and C-terminal ends of the target sequence. The invention of the new arrangement of the primary sequence is “C-intein—C-target-linker—N-target—N-intein” (FIG. 2B). Since the linker sequence is not related to the protein splicing reaction, there is no limitation in introducing any sequence as the linker.

In the present invention, the linker comprises different lengths. The length of the linker can be designed according to the distance of N- and C-terminal ends of individual targets. Since the new strategy of the present invention contains no particular need in using any type of residue in the linker, the linker sequence and length are designed based on distinct requirements. For example, the present invention can introduce an enzymatically cleavable sequence in the linker to control the activation by enzyme treatment. The present invention can introduce a purification tag to assist the target protein purification. The present invention can introduce a binding sequence to promote protein in interaction with other targets. The present invention can use the linker with different lengths to modulate the target protein activity and stability.

To ensure the execution of intein cleavage and ligation, the split site contains a ligation junction that needs to include a certain type of residue providing a side-chain nucleophile (—SH and —OH), such as Cys, Ser or Thr.

The present invention provides an activatable polypeptide comprising, in the N-terminal to C-terminal direction: (1) a C-intein; (2) a C-interleukin; (3) a linker; (4) a N-interleukin and (5) a N-intein, wherein the C-interleukin and the N-interleukin are obtained by splitting a target interleukin, the splitting site is a site of an amino acid with a side-chain nucleophile in the peptide sequence of the target interleukin, and the N-terminus of the C-interleukin contains the amino acid with the side-chain nucleophile.

The present invention also provides a method for producing an activatable polypeptide comprising: preparing a sequence arrangement of peptides to form the activatable polypeptide, wherein the sequence arrangement of peptides comprises, in the N-terminal to C-terminal direction: (1) a C-intein; (2) a C-interleukin; (3) a linker; (4) a N-interleukin; and (5) a N-intein, wherein the C-interleukin and the N-interleukin are obtained by splitting a target interleukin, the splitting site is a site of an amino acid with a side-chain nucleophile in the peptide sequence of the target interleukin, and the N-terminus of the C-interleukin contains the amino acid with the side-chain nucleophile.

In the present invention, the C-terminus of the C-intein connects to the N-terminus of the C-interleukin, the C-terminus of the C-interleukin connects to the N-terminus of the linker, the C-terminus of the linker connects to the N-terminus of the N-interleukin, and the C-terminus of the N-interleukin connects to the N-terminus of the N-intein.

In one embodiment, the C-intein comprises the peptide sequence of SEQ ID NO: 1, and the N-intein comprises the peptide sequence of SEQ ID NO: 2.

In another embodiment, the target interleukin comprises interleukin-2, interleukin-6 or interleukin-15. In the present invention, the interleukin-2 comprises the peptide sequence of SEQ ID No: 3 or 4, the interleukin-15 comprises the peptide sequence of SEQ ID No: 19, and the interleukin-6 comprises the peptide sequence of SEQ ID No: 28. In a preferred embodiment, the target interleukin comprises the peptide sequence of SEQ ID No: 3, 4, 19 or 28.

The sequences of the target interleukins are divided into two fragments: one is the C-interleukin and the other is the N-interleukin. In one embodiment, the C-interleukin comprises the peptide sequence of SEQ ID NO: 5, and the N-interleukin comprises the peptide sequence of SEQ ID NO: 6 when the target interleukin is interleukin-2. In another embodiment, the C-interleukin comprises the peptide sequence of SEQ ID NO: 20, and the N-interleukin comprises the peptide sequence of SEQ ID NO: 21 when the target interleukin is interleukin-15. In one embodiment, the C-interleukin comprises the peptide sequence of SEQ ID NO: 29 or 31, and the N-interleukin comprises the peptide sequence of SEQ ID NO: 30 or 32 when the target interleukin is interleukin-6.

The present invention chooses a split site to separate the sequence of the target interleukin into N-interleukin and C-interleukin. There is a ligation junction in the N terminal end of the C-interleukin. The ligation junction is between the C-intein and the C-interleukin. The function of the ligation junction is used for connecting to the C terminus of the N-interleukin when the C-intein and the N-intein are excised. In the present invention, the split site is chosen to contain a ligation junction that is required to contain the type of residue enabling to provide the side-chain nucleophile, such as —SH and —OH. In one embodiment, the amino acid with the side-chain nucleophile comprises cysteine, serine or threonine. In a preferred embodiment, the amino acid with the side-chain nucleophile is cysteine.

In the present invention, the linker is inserted between the C-interleukin and the N-interleukin. In another embodiment, the peptide sequence of the linker comprises GS repeated sequence or poly G sequence. In a preferred embodiment, the peptide sequence of the linker comprises GSGSGS, GSGSG, GGGGG, GSGS, or GSG.

The length of the linker is depending on the requirement of the influence on interleukin function and stability. In a preferred embodiment, the length of the linker ranges from 2 to 8 amino acids. In a more preferred embodiment, the length of the linker ranges from 3 to 6 amino acids. Since the linker is not related to the cyclization process, there is no limitation in introducing any length and any sequence to be the linker. Through changing the linker, the present invention becomes an effective strategy to modulate interleukin function, such as using different lengths of the linker to enhance or reduce interleukin activity; an enzyme-cleavage sequence to control interleukin function; a designed sequence for enhancing binding; a purification tag for assisting purification.

In the designing IL-2 of the present invention, the C-intein (SEQ ID NO: 1), the C-terminal fragment of IL-2 (SEQ ID NO: 5), the linkers (GSGSG, GGGGG, GSGS, and GSG, respectively), the N-terminal fragment of IL-2 (SEQ ID NO: 6) and the N-intein (SEQ ID NO: 2) are in order for forming the sequence of SEQ ID Nos: 11, 12, 13 and 14 of the IL-2s.

In the designing IL-15 of the present invention, the C-intein (SEQ ID NO: 1), the C-terminal fragment of IL-15 (SEQ ID NO: 20), the linkers (GSGSGS, GSGSG, and GSGS, respectively), the N-terminal fragment of IL-15 (SEQ ID NO: 21) and the N-intein (SEQ ID NO: 2) are in order for forming the sequence of SEQ ID Nos: 22, 23 and 24 of the IL-15s.

In the designing IL-6 of the present invention, the C-intein (SEQ ID NO: 1), the C-terminal fragment of IL-6 (SEQ ID NO: 29 or 31), the linker (GSGSG), the N-terminal fragment of IL-6 (SEQ ID NO: 30 or 32) and the N-intein (SEQ ID NO: 2) are in order for forming the sequence of SEQ ID Nos: 33 and 34 of the IL-6s.

In the new strategy of the present invention, the sequence of the activatable peptide comprises the peptide sequence of SEQ ID No: 11, 12, 13, 14, 22, 23, 24, 33 or 34.

The present invention further provides a cyclized polypeptide, which is in a circular form comprising a C-interleukin; a linker; and a N-interleukin, wherein the N-terminus of the C-interleukin is linked with the C-terminus of the N-interleukin, the N-terminus of the N-interleukin is linked with the C-terminus of the linker, and the N-terminus of the linker is linked with the C-terminus of the C-interleukin, and the C-interleukin and the N-interleukin are obtained by splitting a target interleukin, the splitting site is a site of an amino acid with a side-chain nucleophile in the peptide sequence of the target interleukin, and the N-terminus of the C-interleukin contains the amino acid with the side-chain nucleophile.

The present invention also provides a method for producing a cyclized polypeptide, comprising: (a) providing an activatable polypeptide comprising, in the N-terminal to C-terminal direction: (1) a C-intein; (2) a C-interleukin; (3) a linker; (4) a N-interleukin and (5) a N-intein, wherein the C-interleukin and the N-interleukin are obtained by splitting a target interleukin, the splitting site is a site of an amino acid with a side-chain nucleophile in the peptide sequence of the target interleukin, and the N-terminus of the C-interleukin contains the amino acid with the side-chain nucleophile; and (b) making the activatable polypeptide to be subjected to an intein-mediated protein splicing for allowing splicing of the C-intein and the N-intein and linking of the N-terminus of the C-interleukin to the C-terminus of the N-interleukin to form the cyclized polypeptide.

In one embodiment, the peptide sequence of the linker comprises GSGSGS, GSGSG, GGGGG, GSGS, or GSG.

In another embodiment, the target interleukin comprises interleukin-2, interleukin-15 or interleukin-6. In a preferred embodiment, the sequence of the target interleukin comprises the peptide sequence of SEQ ID No: 3, 4, 19 or 28.

In one embodiment, the sequence of the cyclized polypeptide comprises the peptide sequence of SEQ ID No: 15 to 18, 25 to 27, 35 or 36.

The design of cyclized IL-2, IL-15 and IL-6 create structurally folded and stable interleukins. The present invention promotes the applications of the interleukins in all aspects, such as in regulating immune responses and immune cell migration and proliferation. The features extend the therapeutic usage. The applications of the cyclized interleukins include acting ingredients in cell culture medium to enhance cell growth, supplementary proteins for cell therapy, and protein drugs for cancer therapy.

The above arrangement of sequence in the polypeptide provides feasibility to produce cyclized interleukins, that many interleukins adopt similar structural fold comprising four-helix bundles and N- and C-terminal ends in a proximal position. The split intein (N-intein and C-intein) system is used to prepare the cyclized interleukins.

In conclusion, the cyclized polypeptide of the present invention is in a circular form comprising the peptide sequence of SEQ ID No: 9, 10, 15 to 18, 25 to 27, 35 or 36.

In some embodiments, the interleukin or the target interleukin comprises interleukin-2 (IL-2), interleukin-3 (IL-3), interleukin-4 (IL-4), interleukin-5 (IL-5), interleukin-6 (IL-6), interleukin-7 (IL-7), interleukin-10 (IL-10), interleukin-11 (IL-11), interleukin-12 (IL-12), interleukin-13 (IL-13), interleukin-15 (IL-15), interleukin-16 (IL-16), interleukin-19 (IL-19), interleukin-20 (IL-20), interleukin-21 (IL-21), interleukin-22 (IL-22), interleukin-23 (IL-23), interleukin-24 (IL-24), interleukin-27 (IL-27), interleukin-34 (IL-34) or other protein subtypes with similar four-helix bundle structural fold.

The present invention also provides a pharmaceutical composition comprising the above cyclized polypeptide and a pharmaceutical acceptable carrier or excipient.

In another aspect, the cyclized polypeptide comprising interleukin in the present invention can be co-administered with one or more further therapeutic agents. They can be co-administered simultaneously with such agents (e.g., in a single composition or separately) or can be administered before or after administration of such agents. Such agents can be one or more additional anti-cancer drugs, chemo drugs, radioactive drugs, immune checkpoint inhibitors, or a combination thereof.

After administering, the activatable polypeptide undergoes protein splicing to form the cyclized interleukin for treating diseases or bioindustrial usage.

In the present invention, the intein or the split intein used in the above polypeptide is capable of protein splicing in trans or cis. Inteins of any origin (i.e., naturally occurring inteins or catalytically active naturally occurring or man-made variants thereof) may be employed in the polypeptide described herein. An intein may be of bacterial, yeast, mammalian or viral origin, for example. Accordingly and without wishing to limit the invention to any particular intein, exemplary inteins for use in the polypeptide include: Npu DnaE intein, Ssp DnaB intein, Mxe GyrA intein, CIV RIR1 intein, Ctr VMA intein, Gth DnaB intein, Ppu DnaB intein, Sce VMAAintein, Mfl RecAintein, Ssp DnaE intein, Mle DnaB intein, Mja KIbA intein, Pfu KIbA intein, Mth RIR1 intein, Pfu RIR1-1 intein, Psp-GBD Pol intein, Thy Pol-2 intein, Pfu IF2 intein, Pho Lon intein, Mja r-Gyr intein, Pho RFC intein, Pab RFC-2 intein, Mja RtcB (Mja Hyp-2) intein, Pho VMA intein, Mtu RecA intein, the PI-pfuI intein and the PU-pfu II intein, or artificial trans-splicing variants thereof.

As is well recognized in the art, inteins typically are composed of two domains (termed herein the “N-intein” and “C-intein”) that can be naturally (in the case of the Npu DnaE intein, for example) or non-naturally (i.e., artificially or by recombinant means, for example) present as two different fragments. These intein fragments, when present together, can reconstitute an active intein, and can be used to join two different polypeptides together in trans or in cis. Also as well recognized in the art, inteins may be used to produce cyclic peptides in vivo and in vitro.

Therefore, the present invention provides a method for producing a cyclized polypeptide, comprising providing the above activatable polypeptide, and the above activatable polypeptide is subjected to the intein reaction conditions to produce the cyclized polypeptide. When subjected to suitable in vivo or in vitro intein reaction conditions, the above polypeptide will autocatalyze cyclization of the polypeptide to produce the cyclized polypeptide comprising the interleukin.

The present invention constructs a nucleic acid molecule encoding the above activatable polypeptide. Expression of the nucleic acid molecule in a host system produces the activatable polypeptide that spontaneously splices in the host system to yield the cyclized polypeptide. Therefore, the present invention provides a nucleic acid molecule encoding the above activatable polypeptide. In addition, the present invention also provides an expression vector comprising the nucleic acid molecule. Expression vectors of the invention can be a plasmid, a bacteriophage, a virus, a linear nucleic acid molecule, or other type of vector.

Also within the invention is a host system harboring a nucleic acid molecule of the invention. The host system can be a prokaryote such as a bacterium, an archaebacterium, a eukaryote such as a yeast, a mammalian cell, or a plant cell, an in vitro transcription/translation system, or a cell lysate.

In another aspect, the invention features a method for making the activatable polypeptide. This method includes the steps of: providing an isolated nucleic acid molecule encoding the activatable polypeptide; providing a host system; introducing the isolated nucleic acid molecule into the host system; and expressing the isolated nucleic acid molecule. In one variation, the step of expressing the isolated nucleic acid molecule results in the production of the activatable polypeptide that spontaneously splices in the host system to yield the cyclized polypeptide. This method can also feature the step of purifying the cyclized polypeptide from the host system. Therefore, the present invention further provides a host cell comprising the above activatable polypeptide.

In the classic method, the ligation junction in the first linker (linker 1) prefers to use Cys residue because of its side-chain nucleophile (—SH). However, the additional Cys creates chances to have dimers in solution that are connected through intermolecular disulfide bonds. The dimer formation might affect protein activity. Instead, the new strategy of the present invention has no concern in the intermolecular disulfide bond that no additional Cys is required to be used in the linker.

The cyclized interleukins no matter prepared from the classic method or the new invitation of the present invention can used to replace the usage of interleukin.

### EXAMPLES

The present invention may be implemented in many different forms and should not be construed as limited to the examples set forth herein. The described examples are not limited to the scope of the present invention as described in the claims.

### Example 1: The Design Strategy for Producing the Cyclized Protein

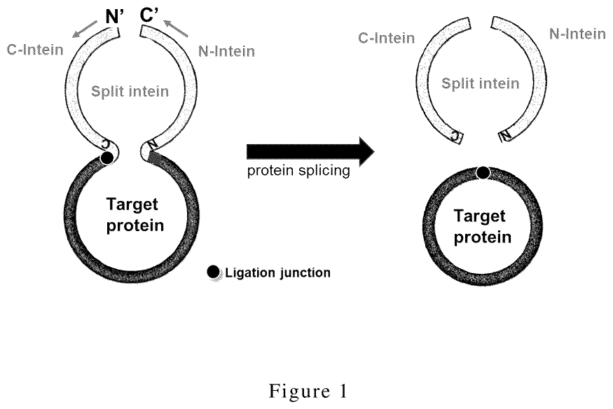

FIG. 1 showed how to use a split intein to produce the cyclic protein. Split intein was derived from one-fragment intein, and the intein sequence was split into two fragments that were N-intein and C-intein. The conjugation of N-intein and C-intein with the target protein resulted in one polypeptide chain. With proper design, the polypeptide could be expressed, and fragments of N-intein and C-intein would perform protein splicing to ligate the target protein in a head-to-tail cyclization.

All active split intein can be used in the present invention. The commonly used inteins include Npu DnaE intein and Ssp DnaB intein.

FIG. 2 showed the two designs for producing cyclic protein.

FIG. 2A showed the scheme of the classic method for producing cyclized protein. The arrangement of activatable polypeptide was “C-intein—linker 1—target sequence—linker 2—N-intein”. There was a restriction of linker 1. To ensure the cyclization was feasible, a reside containing the side-chain nucleophile (—SH and —OH) should be introduced in the ligation junction, the N-terminal site of linker 1.

FIG. 2B showed the scheme of the present invention for producing cyclized protein. The target sequence was split into two fragments, N-target and C-target. The “N-target” represented the N-terminal portion of the target sequence, and the “C-target” represented the C-terminal portion of the target sequence. The split site in the target sequence was properly selected. The arrangement of the activatable polypeptide provided by the present invention was “C-intein—C-target—linker—N-target—N-intein”.

In the current new design of the present invention, residue in the target sequence, which could provide side-chain nucleophile, such as —SH and —OH, would be selected to be the ligation junction, and the target sequence was split into two fragments according to the selection of the residue. The residue (ligation junction) became the first residue of C-target. The residue was responsible for executing the following transesterification reaction. The residue made the cyclization feasible.

In the current new design of the present invention, there was a linker between C-target and N-target. The linker was not involved in protein backbone cyclization reaction. The linker length and sequence could be arbitrarily chosen to assist target protein folding. There is no restriction for linker sequence and length.

### Example 2: The Production of Cyclized Interleukin-2 (IL-2)

The cyclized IL-2 invention proved the feasibility of using the present invention to prepare cyclized interleukin.

IL-2 was found in 1980s and applied to propagate activated T cells. In 1990s, IL-2 was capable of treating the metastatic renal cell carcinoma and the metastatic melanoma. In 2000s, several drugs comprising IL-2 sequence had been well-developed, such as Proleukin, Bioleukin etc.

In view of the above technical circumstances, the present invention provided a method to produce cyclized polypeptide, which specifically bound to an example of interleukin family protein, interleukin 2 (IL-2). For preparing the cyclized polypeptide, the peptide fragments used in the present invention comprised a C-terminal fragment of split intein (C-intein, SEQ ID NO: 1), a N-terminal fragment of split intein (N-intein, SEQ ID NO: 2), a C-terminal fragment of IL-2 (C-target, SEQ ID NO: 5) and a N-terminal fragment of IL-2 (N-target, SEQ ID NO: 6). The native amino acid sequence of IL-2 was shown in SEQ ID NO: 3, and IL-2 was split into the N-terminal fragment of IL-2 and the C-terminal fragment of IL-2, where the split site in the IL-2 sequence was introduced at the junction between residues Q57 and C58 (sequence number referencing to SEQ ID NO: 3).

Unless otherwise noted, the designed proteins were expressed by E. coli expression vectors that harbored their DNA sequences. The oligonucleotides were prepared by DNA synthesis. Plasmids (pET-28a(+)) and BL21(DE3) competent cells used for expression were from Novagen.

In the present invention, a cyclized polypeptide specifically bound to an IL-2 and formed the activatable cyclized IL-2. The present invention presented the cyclized IL-2s with two forms. The first form was the cyclized IL-2s derived from the classic arrangement that the amino acid sequence was as shown in SEQ ID NOs: 7 and 8. The represented method was able to produce cyclized IL-2 and adopted the arrangement of “C-intein—linker 1—target sequence—linker 2—N-intein”, wherein the sequence of the linker 1 is CFN, and the sequence of the linker 2 is GS, respectively. The classic arrangement of the sequence containing native IL-2 sequence was as shown in SEQ ID NO: 7. The cyclized IL-2 sequence containing the native IL-2 sequence prepared by the classic arrangement was shown in SEQ ID NO: 9 (IL-2 C5A). An alternative sequence containing one mutation at position 125 (C125S) in the native IL-2 sequence to reduce protein aggregation property is shown in SEQ ID NO: 4. The mutation prevented the dimer formation through intermolecular disulfide bond. The classic arrangement of the sequence containing IL-2 with mutation was as shown in SEQ ID NO: 8. The cyclized IL-2 sequence containing the sequence of the IL-2 with mutation prepared by the classic arrangement was shown in SEQ ID NO: 10 (IL-2 C5B).

In the embodiment of the activatable sequence of IL-2 prepared by the classic method, CFN and GS were used as the first and second linkers, respectively.

The second form was based on the new arrangement of IL-2 comprised an amino acid sequence as shown in SEQ ID NOs: 11-14. The method to produce cyclic IL-2 adopted the new arrangement of “C-intein—C-target—linker—N-target—N-intein”, wherein any desired linker sequence could be introduced. The arrangement proposed by the present invention produced cyclized IL-2, wherein the linker could be designed without restriction. Therefore, the cyclized IL-2s prepared by the present invention were as shown in SEQ ID NOs: 15 (IL-2 G5A), 16 (IL-2 G5B), 17 (IL-2 G4A) and 18 (IL-2 G3A).

TABLE 1
Sequence used in the present invention for
preparing cyclized IL-2s

C-intein
SEQ ID NO: 1
MIKIATRKYLGKQNVYDIGVERDHNFALKNGFIASN
N-intein
SEQ ID NO: 2
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PNLEHHHHHH
Native IL-2 sequence
SEQ ID NO: 3
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT
IL-2 sequence with a mutation (C125S)
SEQ ID NO: 4
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFSQSIISTLT
C-terminal fragment of IL-2 sequence
SEQ ID NO: 5
CLEEELKPLEEVLNLAQSKNFHLRPRDLISNINVIVLELKGSETTFMCEY
ADETATIVEFLNRWITFCQSIISTLT
N-terminal fragment of IL-2 sequence
SEQ ID NO: 6
APTSSSTKKTQLQLEHLLLDLQMILNGINNYKNPKLTRMLTFKFYMPKKA
TELKHLQ
Classic arrangement of IL-2 C5A
SEQ ID NO: 7
MIKIATRKYLGKQNVYDIGVERDHNFALKNGFIASNCFNAPTSSSTKKTQ
LQLEHLLLDLQMILNGINNYKNPKLTRMLTFKFYMPKKATELKHLQCLEE
ELKPLEEVLNLAQSKNFHLRPRDLISNINVIVLELKGSETTFMCEYADET
ATIVEFLNRWITFCQSIISTLTGSCLSYETEILTVEYGLLPIGKIVEKRI
ECTVYSVDNNGNIYTQPVAQWHDRGEQEVFEYCLEDGSLIRATKDHKFMT
VDGQMLPIDEIFERELDLMRVDNLPNLEHHHHHH
Classic arrangement of IL-2 C5B with
mutation (C125S)
SEQ ID NO: 8
MIKIATRKYLGKQNVYDIGVERDHNFALKNGFIASNCFNAPTSSSTKKTQ
LQLEHLLLDLQMILNGINNYKNPKLTRMLTFKFYMPKKATELKHLQCLEE
ELKPLEEVLNLAQSKNFHLRPRDLISNINVIVLELKGSETTFMCEYADET
ATIVEFLNRWITFSQSIISTLTGSCLSYETEILTVEYGLLPIGKIVEKRI
ECTVYSVDNNGNIYTQPVAQWHDRGEQEVFEYCLEDGSLIRATKDHKFMT
VDGQMLPIDEIFERELDLMRVDNLPNLEHHHHHH
Cyclized IL-2 from SEQ ID NO: 7: IL-2 C5A
SEQ ID NO: 9
CFNAPTSSSTKKTQLQLEHLLLDLQMILNGINNYKNPKLTRMLTFKFYMP
KKATELKHLQCLEEELKPLEEVLNLAQSKNFHLRPRDLISNINVIVLELK
GSETTFMCEYADETATIVEFLNRWITFCQSIISTLTGS
Cyclized IL-2 from SEQ ID NO: 8: IL-2 C5B
SEQ ID NO: 10
CFNAPTSSSTKKTQLQLEHLLLDLQMILNGINNYKNPKLTRMLTFKFYMP
KKATELKHLQCLEEELKPLEEVLNLAQSKNFHLRPRDLISNINVIVLELK
GSETTFMCEYADETATIVEFLNRWITFSQSIISTLTGS
The activatable polypeptide of IL-2 G5A
of the present invention
SEQ ID NO: 11
MIKIATRKYLGKQNVYDIGVERDHNFALKNGFIASNCLEEELKPLEEVLN
LAQSKNFHLRPRDLISNINVIVLELKGSETTFMCEYADETATIVEFLNRW
ITFCQSIISTLTGSGSGAPTSSSTKKTQLQLEHLLLDLQMILNGINNYKN
PKLTRMLTFKFYMPKKATELKHLQCLSYETEILTVEYGLLPIGKIVEKRI
ECTVYSVDNNGNIYTQPVAQWHDRGEQEVFEYCLEDGSLIRATKDHKFMT
VDGQMLPIDEIFERELDLMRVDNLPNLEHHHHHH
The activatable polypeptide of IL-2 G5B
of the present invention
SEQ ID NO: 12
MIKIATRKYLGKQNVYDIGVERDHNFALKNGFIASNCLEEELKPLEEVLN
LAQSKNFHLRPRDLISNINVIVLELKGSETTFMCEYADETATIVEFLNRW
ITFCQSIISTLTGGGGGAPTSSSTKKTQLQLEHLLLDLQMILNGINNYKN
PKLTRMLTFKFYMPKKATELKHLQCLSYETEILTVEYGLLPIGKIVEKRI
ECTVYSVDNNGNIYTQPVAQWHDRGEQEVFEYCLEDGSLIRATKDHKFMT
VDGQMLPIDEIFERELDLMRVDNLPNLEHHHHHH
The activatable polypeptide of IL-2 G4A
of the present invention
SEQ ID NO: 13
MIKIATRKYLGKQNVYDIGVERDHNFALKNGFIASNCLEEELKPLEEVLN
LAQSKNFHLRPRDLISNINVIVLELKGSETTFMCEYADETATIVEFLNRW
ITFCQSIISTLTGSGSAPTSSSTKKTQLQLEHLLLDLQMILNGINNYKNP
KLTRMLTFKFYMPKKATELKHLQCLSYETEILTVEYGLLPIGKIVEKRIE
CTVYSVDNNGNIYTQPVAQWHDRGEQEVFEYCLEDGSLIRATKDHKFMTV
DGQMLPIDEIFERELDLMRVDNLPNLEHHHHHH
The activatable polypeptide of IL-2 G3A
of the present invention
SEQ ID NO: 14
MIKIATRKYLGKQNVYDIGVERDHNFALKNGFIASNCLEEELKPLEEVLN
LAQSKNFHLRPRDLISNINVIVLELKGSETTFMCEYADETATIVEFLNRW
ITFCQSIISTLTGSGAPTSSSTKKTQLQLEHLLLDLQMILNGINNYKNPK
LTRMLTFKFYMPKKATELKHLQCLSYETEILTVEYGLLPIGKIVEKRIEC
TVYSVDNNGNIYTQPVAQWHDRGEQEVFEYCLEDGSLIRATKDHKFMTVD
GQMLPIDEIFERELDLMRVDNLPNLEHHHHHH
The cyclized IL-2 from SEQ ID NO: 11:
IL-2 G5A
SEQ ID NO: 15
CLEEELKPLEEVLNLAQSKNFHLRPRDLISNINVIVLELKGSETTFMCEY
ADETATIVEFLNRWITFCQSIISTLTGSGSGAPTSSSTKKTQLQLEHLLL
DLQMILNGINNYKNPKLTRMLTFKFYMPKKATELKHLQ
The cyclized IL-2 from SEQ ID NO: 12:
IL-2 G5B
SEQ ID NO: 16
CLEEELKPLEEVLNLAQSKNFHLRPRDLISNINVIVLELKGSETTFMCEY
ADETATIVEFLNRWITFCQSIISTLTGGGGGAPTSSSTKKTQLQLEHLLL
DLQMILNGINNYKNPKLTRMLTFKFYMPKKATELKHLQ
The cyclized IL-2 from SEQ ID NO: 13:
IL-2 G4A
SEQ ID NO: 17
CLEEELKPLEEVLNLAQSKNFHLRPRDLISNINVIVLELKGSETTFMCEY
ADETATIVEFLNRWITFCQSIISTLTGSGSAPTSSSTKKTQLQLEHLLLD
LQMILNGINNYKNPKLTRMLTFKFYMPKKATELKHLQ
The cyclized IL-2 from SEQ ID NO: 14:
IL-2 G3A
SEQ ID NO: 18
CLEEELKPLEEVLNLAQSKNFHLRPRDLISNINVIVLELKGSETTFMCEY
ADETATIVEFLNRWITFCQSIISTLTGSGAPTSSSTKKTQLQLEHLLLDL
QMILNGINNYKNPKLTRMLTFKFYMPKKATELKHLQ

In Table 1, the sequences with underlines were designed by the present invention for cyclized IL-2s.

In the embodiment of activatable sequences of IL-2 in the present invention, the present invention introduced linkers of GSGSG (SEQ ID NO: 11, IL-2 G5A), GGGGG (SEQ ID NO: 12, IL-2 G5B), GSGS (SEQ ID NO: 13, IL-2 G4A), and GSG (SEQ ID NO: 14, IL-2 G3A) as examples. The present invention needed emphasize that the design of linker was not limited in the reported linkers. Arbitrarily amino acid sequence and length could be used to replace the current mentioned linker for preparing cyclized version of IL-2, according to different requirements.

The plasmids harboring the designs of cyclized version of IL-2 were transformed into BL21(DE3) competent Cells. The intein chosen for preparing cyclized IL-2 was Npu DnaE intein. The protein expression was induced by adding 1 mM IPTG (Isopropyl β-D-1-thiogalactopyranoside). After induction for 6-10 hours at 37° C., the present design brought significant expression that IL-2 was readily excised from Npu DnaE intein after expression. The present invention confirmed that the IL-2 was already cyclized through mass spectrum and NMR methods.

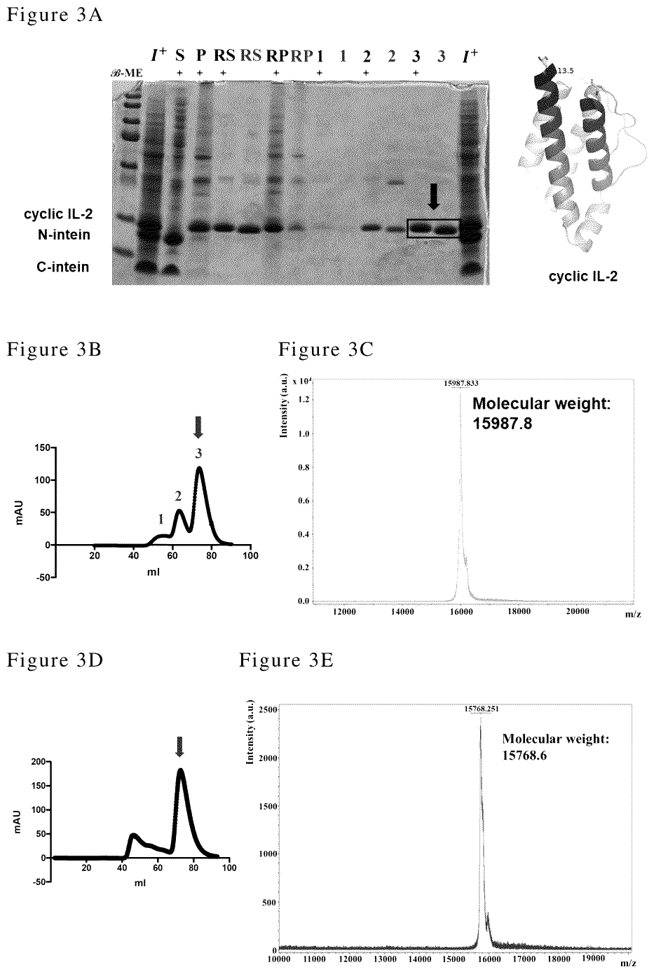

FIGS. 3A-3E showed the purification and characterization of the cyclized IL-2. The currently designed cyclized IL-2s (SEQ ID NOs: 9-10 and 15-18) showed great consistency in the purification in the present invention. All cyclized IL-2s had the same behavior during expression and purification. Two variants of IL-2 C5A (from the classic method) and IL-2 G5A (from the new arrangement of the present invention) were selected to represent the purification results of cyclized IL-2s.

FIG. 3A showed SDS-PAGE analysis confirming the expression of cyclized IL-2 from the present invention. IL-2 was readily cyclized and excised from intein after protein expression that the bands corresponding to cyclized IL-2, N-intein and C-intein were detected in the SDS-PAGE analysis. I⁺: IPTG induction; S: supernatant of cell lysis; P: pellet of cell lysis; RS: supernatant after refolding from pellet; RP: precipitation after refolding from pellet; 1-3: the respective fractions separated by FPLC size-exclusion chromatography (FIG. 3B). The samples prepared with adding β-mercaptoethanol was denoted by “+”. The bands representing the purified cyclized IL-2 was indicated by box and arrow. The presence of β-mercaptoethanol reduced disulfide bond in IL-2 and caused slightly different mobility shift in the SDS-PAGE analysis.

FIG. 3B showed the FPLC purification profile of the cyclized IL-2 C5A. The cyclized IL-2 was purified by using a Hiload 26/60 Superdex 75 size-exclusion column with flow rate of 1 ml/min. The fraction 1 represented the impurity and the fraction 2 was IL-2 dimer. The fraction 3 represented the cyclized IL-2 monomer, indicated by arrow.

The cyclized IL-2 prepared by the classic method showed a disadvantage. The linker 1 contained residue Cys, and the residue acted as the ligation junction. The presence of the Cys residue created a chance to form intermolecular disulfide bond. Therefore, it made disulfide-bond-linked dimer, as indicated in the fraction 2 in FIG. 3B.

FIG. 3C showed the mass result of the cyclized IL-2 C5A. The mass result supported the purity of the cyclized IL-2 C5A, and the measured molecular weight confirmed the structure containing the “GSCFN” linker.

FIG. 3D showed the FPLC purification profile of the cyclized IL-2 G5A. The cyclized IL-2 was purified by using a Hiload 26/60 Superdex 75 size-exclusion column with flow rate of 1 ml/min. The fraction represented the cyclized IL-2 monomer, indicated by arrow.

FIG. 3E showed the mass result of the cyclized IL-2 G5A. The mass result supported the purity of the cyclized IL-2, and the measured molecular weight confirmed the structure containing the “GSGSG” linker.

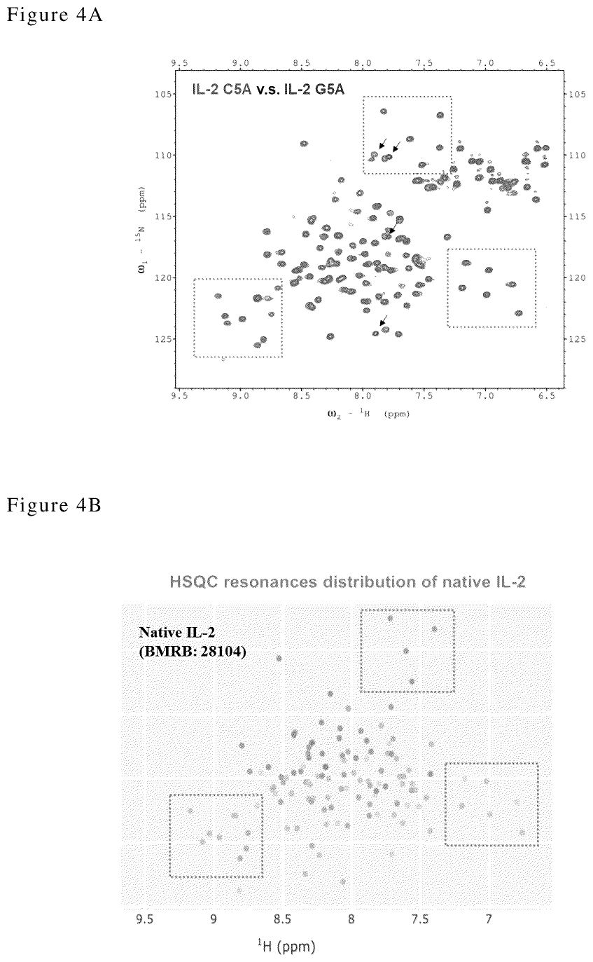

FIG. 4A showed ¹H-¹⁵N Heteronuclear single quantum coherence (HSQC) spectra of cyclized IL-2 C5A and G5A. The two cases respectively represented the cases cyclized by the classic method and the new arrangement in the present invention. The spectrum was acquired on a BRUKER 850 MHz NMR machine at 298K and the sample of 0.1 mM protein was used. The sample buffer was 20 mM sodium phosphate buffer, pH 7.4. The high spectral similarity indicated the same structure between the two cyclized IL-2s and the dispersive resonances indicated the well-folded structures. The cyclized IL-2s, prepared from the present invention, adopted the same conformation, no matter that prepared from the new arrangement or the classic method.

FIG. 4B showed the HSQC resonance distribution of native IL-2 based on deposited chemical shifts in BMRB (code 28104). Comparing to the resonances of native IL-2, the two cyclized IL-2s had extremely similar resonance distribution.

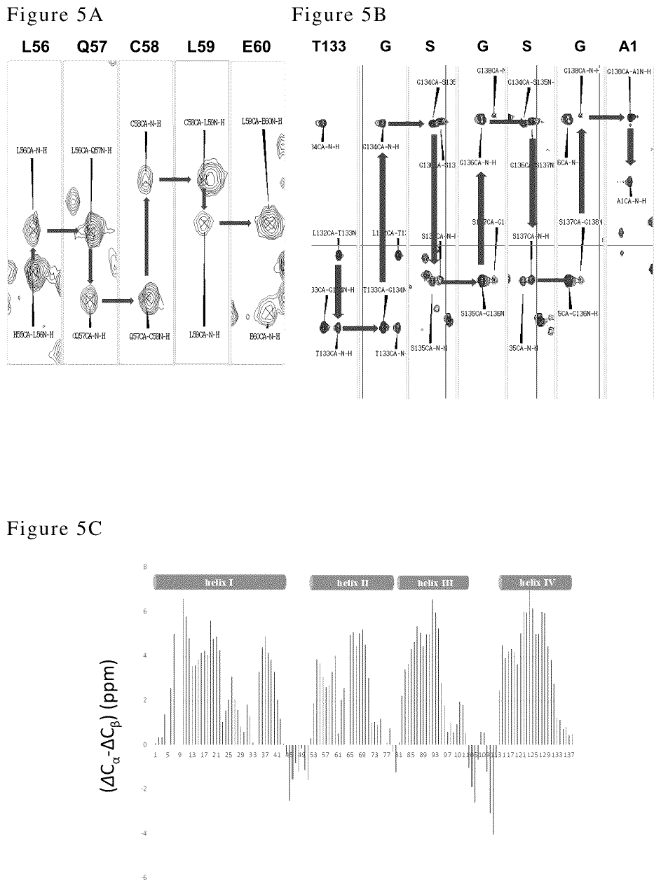

FIG. 5 showed the strip plots of NMR 3D HNCA experiment of IL-2 G5A. The results reported the quality of backbone sequential assignment. The sequential assignment was represented from the residue L56 to residue E60, where the split junction was introduced between residues Q57 and C58 (FIG. 5A) and from residue T133 to Al where a GSGSG linker connected the N- and C-terminal ends (FIG. 5B). The successful sequential assignment revealed the backbone connection, therefore proving the success of cyclization. The NMR condition was with a buffer of 20 mM sodium phosphate, pH 6, 150 mM NaCl. The 3D spectra are acquired on BRUKER 600 MHz NMR machine at 303K.

The HSQC spectrum and backbone sequential assignment permitted to establish the backbone chemical shifts of NH, N, C_{α}, C_{β} and CO. The chemical shifts of C_{α} and C_{β} confirms the facts of that: (i) four helices in the cyclized IL-2, matching the X-ray structure (FIG. 5C); (ii) two oxidized Cys residues (C58 C_{β}=40.39 ppm and C105 C_{β}=45.75 ppm) forming a disulfide linkage; (iii) one reduced Cys residue (C125 C_{β}=27.84 ppm); and (iv) the head-to-tail connection for cyclizing IL-2 backbone.

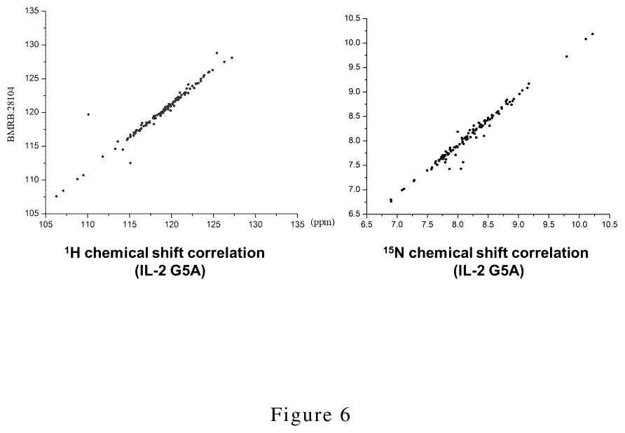

FIG. 6 showed the correlation between experimental chemical shifts (¹H and ¹⁵N) of IL-2 G5A and reported chemical shifts (¹H and ¹⁵N) of native IL-2 (BMRB code 28104). Both assignments were finished under the condition of pH 6.0. The linear correlations in ¹H and ¹⁵N chemical shifts indicated that IL-2 G5A adopted the same structure to native IL-2. Only few residues that distribute near the linker region had different chemical shifts, such as 1122, T131, L132 and T133.

### Example 3: The Cyclized IL-2 Adopts Stable Structure in Solution

The cyclized IL-2s had the same structure compared to native IL-2 structure. The cyclized IL-2 had a higher melting temperature for structure, indicating cyclization enhances their structural stability.

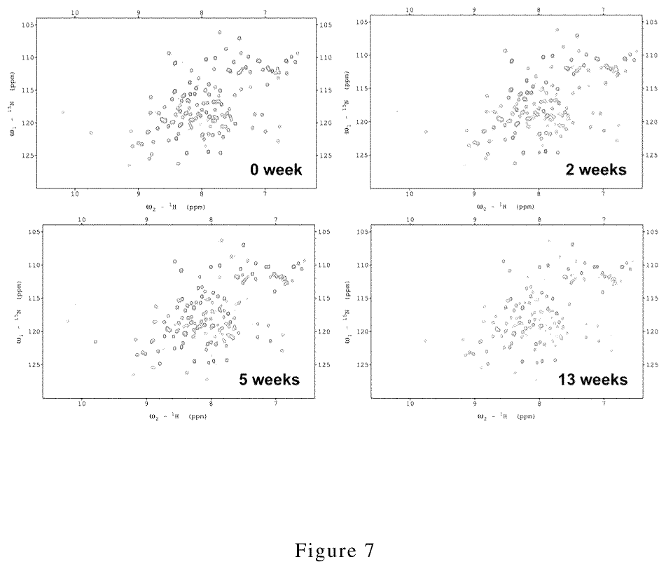

FIG. 7 showed ¹H-¹⁵N HSQC spectra of cyclized IL-2 G5A acquired under different storage times. The samples were freshly prepared (0 week) or stored for 2, 5 or 13 weeks at 4° C., respectively. The IL-2 NMR sample had concentrated to 0.1 mM and was acquired at 298K on a BRUKER 850 MHz NM4R machine. The sample buffer was 20 mM sodium phosphate buffer, pH 7.4. The similarity between HSQCs of the cyclized IL-2 G5A indicated the structural stability. No conformational denaturation or degradation occurred. The result indicated the cyclized IL-2 solution can be stored at 4° C. for over 13 weeks.

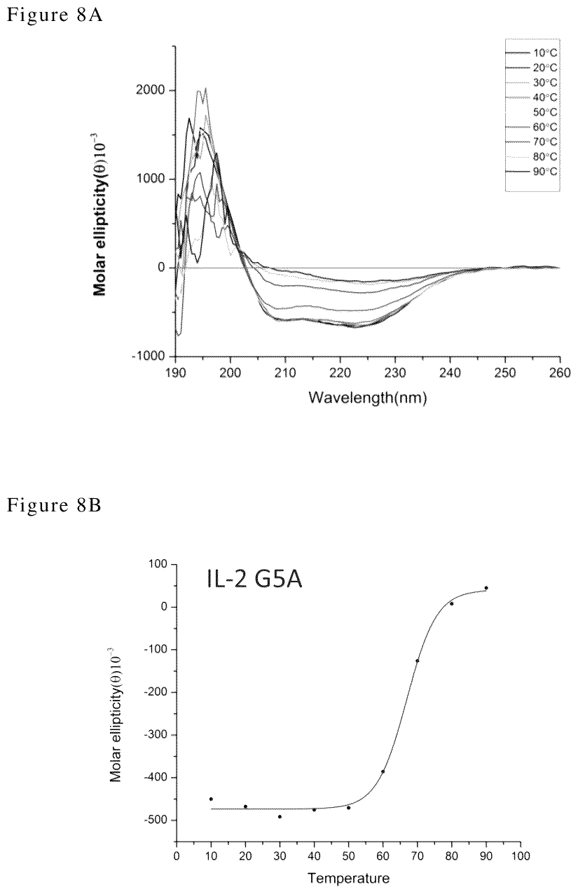

FIG. 8 showed the thermal denaturation tendency, monitored by circular dichroism (CD) spectroscopy. FIG. 8A showed the CD profiles of the cyclized IL-2 G5A acquired under different temperatures (from 10° C. to 90° C. in steps of 10MC) and 10 μM protein sample was used. The sample buffer was 10 mM sodium phosphate buffer, pH 7.4. The profiles in low temperatures indicated that the secondary structure of IL-2 was mainly α-helices. The structural helicity was reduced upon the temperature was increased, indicating denaturation. FIG. 8B showed the thermal denaturation curve of the cyclized IL-2 G5A, monitored at one single wavelength of 208 nm. The melting temperature of the cyclized IL-2 was measured to 76° C.

All cyclized IL-2s had stable protein structure. IL-2 C5A and CB, cyclized through the classic method, had melting temperature of 62° C. and 74° C., respectively. IL-2 GA, G5B, G4A and G3A, cyclized through the new arrangement of the present invention, had melting temperature from 76° C. to 64° C. The details were listed in Table 2. Specifically, the linker with 5-residue length showed the highest melting temperature among the cyclized IL-2.

TABLE 2
The melting temperature of cyclized IL-2 and their abilities
(ED₅₀) to induce proliferation in CTLL-2 cells

| | | Melting | ED₅₀ of | |
|---|---|---|---|---|
| | Melting | temperature of | CTLL-2 cell | The reported ED₅₀ |
| | temperature | native protein | proliferation | of native protein |
| IL-2 C5A | 62° C. | <50° C. | 0.046 nM | 0.0025-0.16 nM |
| (SEQ ID NO: 9) | | | | |
| IL-2 C5B | 74° C. | | 0.017 nM | |
| (SEQ ID NO: | | | | |
| 10) | | | | |
| IL-2 G5A | 76° C. | | 0.013 nM | |
| (SEQ ID NO: | | | | |
| 15) | | | | |
| IL-2 G5B | 74° C. | | — | |
| (SEQ ID NO: | | | | |
| 16) | | | | |
| IL-2 G4A | 67° C. | | 0.046 nM | |
| (SEQ ID NO: | | | | |
| 17) | | | | |
| IL-2 G3A | 64° C. | | 0.033 nM | |
| (SEQ ID NO: | | | | |
| 18) | | | | |

### Example 4: The Long-Lasting Active IL-2

The cyclized IL-2s had long-lasting activity in enhancing cell proliferation.

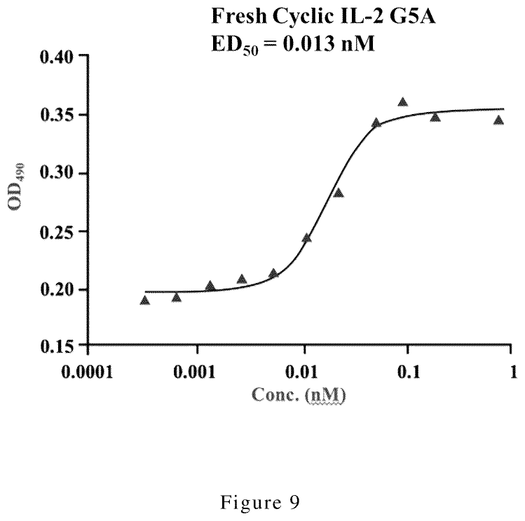

FIG. 9 showed that the activity of cyclized IL-2 G5A was measured in a cell proliferation assay. The experiment was conducted in using CTLL-2 mouse cytotoxic T cells. The IL-2 was prepared at 100 μg/mL in sterile PBS buffer and added into cell culture medium with desired concentrations. The measurement used colorimetric MTT (3-(4,5-dimethylthiazol-2-yl)-2,5-diphenyltetrazolium bromide) assay to measure the number of CTLL-2 cells, reflecting the tendency of cell proliferation. The freshly prepared IL-2 was used in this measurement and the ED₅₀ (50% effective dosage) of IL-2 G5A was 0.013 nM. Table 3 showed the ED₅₀ of the cyclized IL-2 C5B. The ED₅₀ values kept the same range (0.017-0.019 nM) for the samples of 5-week, 10-week, 15-week storage. The ED₅₀ value had slightly increased (0.027 nM) for the sample of 20-week storage. The cyclized IL-2 showed prolonged activity even after the storage period over 20 weeks.

TABLE 3
The ED₅₀ of the cyclized IL-2 C5B

| Cyclized | ED₅₀ | |
|---|---|---|
| IL-2 C5B | (nM) | R² |
| Fresh prepared | 0.017 | 0.987 |
| After 5 weeks | 0.018 | 0.990 |
| After 10 weeks | 0.018 | 0.992 |
| After 15 weeks | 0.019 | 0.992 |
| After 20 weeks | 0.027 | 0.991 |

All cyclized IL-2s designed from the present invention had the prolonged activity. The measured ED₅₀ values were comparable to that of native IL-2. The details of ED₅₀ values were listed in Table 2. The determined ED₅₀ values of cyclized IL-2s were between 0.013-0.046 nM.

### Example 5: The Invention of Cyclized IL-15 with Using Different Linker Lengths

Cyclized IL-15 invention was to prove the feasibility of using different linker lengths to design activatable polypeptide for interleukin.

The native amino acid sequence of IL-15 was as shown in SEQ ID NO: 19. The split site was introduced at the junction between residues K41 and C42 (sequence number referencing to SEQ ID NO: 19) to produce the split IL-15 sequences comprised a C-terminal fragment (SEQ ID NO: 20) and a N-terminal fragment (SEQ ID NO: 21). The intein chosen for preparing cyclized IL-15 was Npu DnaE intein.

The new arrangements of IL-15 in the present invention were shown in SEQ ID NOs: 22 to 24. The cyclized IL-15 of the present invention comprised the amino acid sequences as shown in SEQ ID NO: 25 (IL-15 G6A), SEQ ID NO: 26 (IL-15 G5A) and SEQ ID NO: 27 (IL-15 G4A). Because of the advantage of the present invention, it respectively introduced different linkers with different lengths of GSGSGS, GSGSG and GSGS as examples. Plasmids (pET-28a(+)) was used to harbor the genes and BL21(DE3) competent cells were used for the expression of cyclized IL-15s. The sequences of IL-15s used in the present invention were shown in Table 4.

TABLE 4
Sequence used in the present invention
for preparing cyclized IL-15s

Native IL-15 sequence
SEQ ID NO: 19
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS
C-terminal fragment of IL-15 sequence
SEQ ID NO: 20
CFLLELQVISLESGDASIHDTVENLIILANNSLSSNGNVTESGCKECEEL
EEKNIKEFLQSFVHIVQMFINTS
N-terminal fragment of IL-15 sequence
SEQ ID NO: 21
NWVNVISDLKKIEDLIQSMHIDATLYTESDVHPSCKVTAMK
The activatable polypeptide of IL-15
G6A of the present invention
SEQ ID NO: 22
MIKIATRKYLGKQNVYDIGVERDHNFALKNGFIASN
CFLLELQVISLESGDASIHDTVENLIILANNSLSSNGNVTESGCKECEEL
EEKNIKEFLQSFVHIVQMFINTSGSGSGSNWVNVISDLKKIEDLIQSMHI
DATLYTESDVHPSCKVTAMKCLSYETEILTVEYGLLPIGKIVEKRIECTV
YSVDNNGNIYTQPVAQWHDRGEQEVFEYCLEDGSLIRATKDHKFMTVDGQ
MLPIDEIFERELDLMRVDNLPNLEHHHHHH
The activatable polypeptide of IL-15
G5A of the present invention
SEQ ID NO: 23
MIKIATRKYLGKQNVYDIGVERDHNFALKNGFIASNCFLLELQVISLESG
DASIHDTVENLIILANNSLSSNGNVTESGCKECEELEEKNIKEFLQSFVH
IVQMFINTSGSGSGNWVNVISDLKKIEDLIQSMHIDATLYTESDVHPSCK
VTAMKCLSYETEILTVEYGLLPIGKIVEKRIECTVYSVDNNGNIYTQPVA
QWHDRGEQEVFEYCLEDGSLIRATKDHKFMTVDGQMLPIDEIFERELDLM
RVDNLPNLEHHHHHH
The activatable polypeptide of IL-15
G4A of the present invention
SEQ ID NO: 24
MIKIATRKYLGKQNVYDIGVERDHNFALKNGFIASNCFLLELQVISLESG
DASIHDTVENLIILANNSLSSNGNVTESGCKECEELEEKNIKEFLQSFVH
IVQMFINTSGSGSNWVNVISDLKKIEDLIQSMHIDATLYTESDVHPSCKV
TAMKCLSYETEILTVEYGLLPIGKIVEKRIECTVYSVDNNGNIYTQPVAQ
WHDRGEQEVFEYCLEDGSLIRATKDHKFMTVDGQMLPIDEIFERELDLMR
VDNLPNLEHHHHHH
The cyclized IL-15 G6A from SEQ ID NO: 22
SEQ ID NO: 25
CFLLELQVISLESGDASIHDTVENLIILANNSLSSNGNVTESGCKECEEL
EEKNIKEFLQSFVHIVQMFINTSGSGSGSNWVNVISDLKKIEDLIQSMHI
DATLYTESDVHPSCKVTAMK
The cyclized IL-15 G5A from SEQ ID NO: 23
SEQ ID NO: 26
CFLLELQVISLESGDASIHDTVENLIILANNSLSSNGNVTESGCKECEEL
EEKNIKEFLQSFVHIVQMFINTSGSGSGNWVNVISDLKKIEDLIQSMHID
ATLYTESDVHPSCKVTAMK
The cyclized IL-15 G4A from SEQ ID NO: 24
SEQ ID NO: 27
CFLLELQVISLESGDASIHDTVENLIILANNSLSSNGNVTESGCKECEEL
EEKNIKEFLQSFVHIVQMFINTSGSGSNWVNVISDLKKIEDLIQSMHIDA
TLYTESDVHPSCKVTAMK

In Table 4, the sequences with underlines were designed by the present invention for cyclized IL-15s.

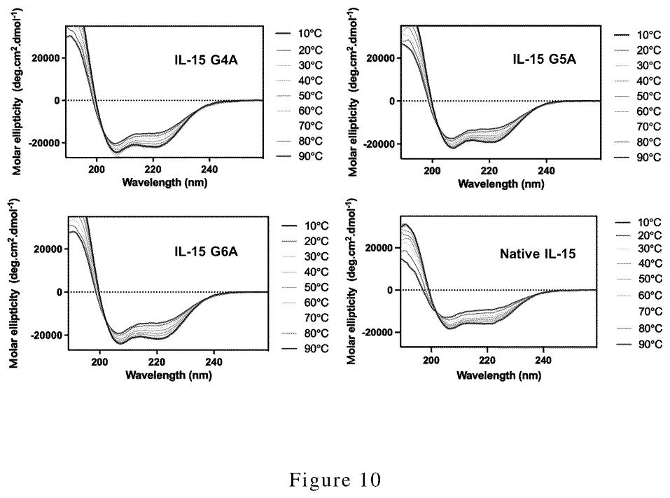

FIG. 10 showed the structural stability of cyclized IL-15s and native IL-15, monitored by circular dichroism (CD) spectra at different temperatures. The four IL-15s showed similar α-helical structures as demonstrated by CD spectroscopy. The increase of temperature reduced the content of helical structure. Notably, the cyclized IL-15s showed similar behavior to native IL-15 but the cyclized IL-15s had higher molar ellipticity, indicating better α-helical secondary structures, confirming that the structural stability was derived from protein cyclization.

Table 5 showed the cyclized IL-15 G6A had the highest melting temperature among the three cyclized IL-15s. The melting temperature of IL15-G6A was 8 degrees higher than native IL-15. The screening of different linker lengths could assist in designing cyclized IL-15 with better stability. The example showed the advantage that the present invention could introduce any linker with desired length.

In addition, Table 5 summarized the melting temperature of cyclized IL-15 and their abilities to induce proliferation in CTLL-2 cells. The determined ED₅₀ values of cyclized IL-15s were ˜0.007 nM.

TABLE 5
The melting temperature and ED₅₀ of cyclized IL-15

| | | Melting | | |
|---|---|---|---|---|
| | Melting | temperature of | | |
| | temperature | native protein | ED₅₀ | The reported ED₅₀ |
| IL-15 G6A | 41° C. | 35° C. | 0.006 nM | 0.05-0.2 nM |
| IL-15 G5A | 42° C. | | 0.007 nM | |
| IL-15 G4A | 43° C. | | 0.008 nM | |

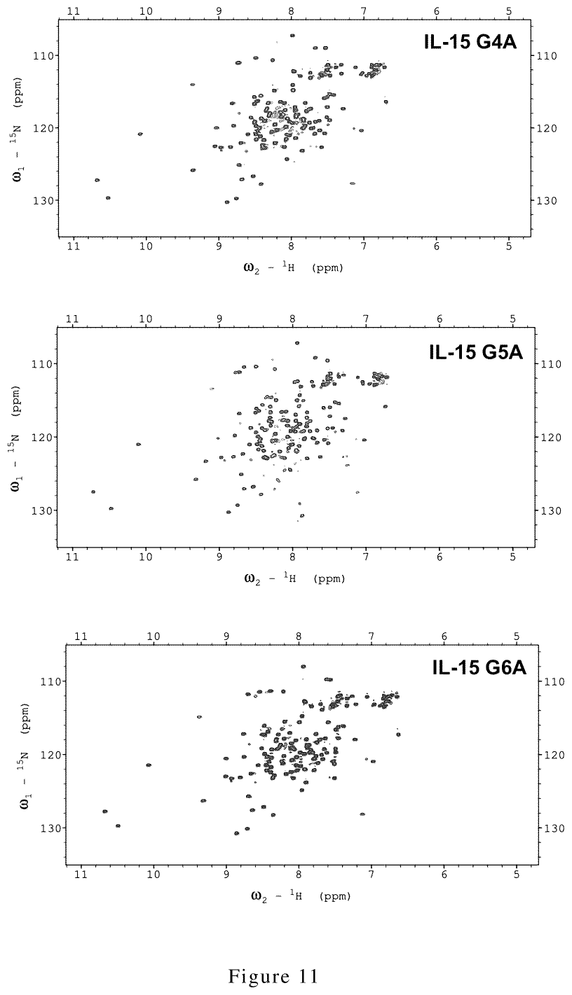

FIG. 11 showed the HSQC spectra of cyclized IL-15 G4A, G5A and G6A. The dispersive resonances indicated a well-folded structure for the three cyclized IL-15. The experiments reported that the cyclized interleukin could maintain the folded structure with the proper length of linker. The length of linker could modulate protein structure. The NMR condition was with a buffer of 25 mM Tris, pH 6.5, 120 mM NaCl and temperature at 298K. The dispersive resonances allowed finishing backbone sequential assignment. The chemical shifts confirmed the facts of that: (i) four helices in the cyclized IL-15, matching the X-ray structure; (ii) four Cys residues all adopting oxidized states by forming two intramolecular disulfide bonds; (iii) the head-to-tail connection for cyclizing IL-15 backbone; and (iv) highly dynamic structural property as evidenced by the nearly 30 missing resonances in HSQC.

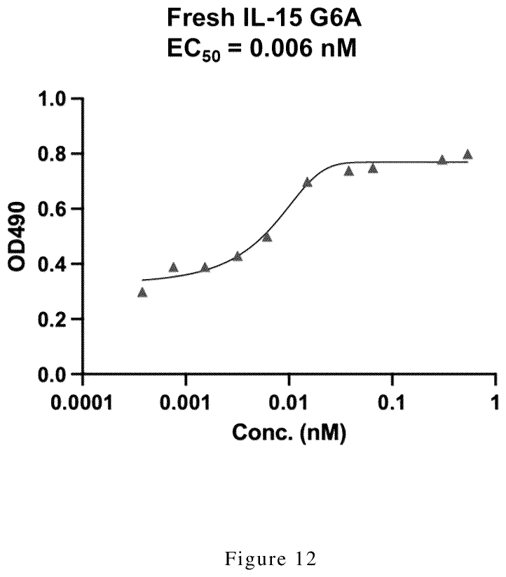

FIG. 12 showed cell proliferation activity of cyclized IL-15 G6A where IL-15 G6A was selected as a representative case because of its structural stability. The experiment was conducted in using CTLL-2 mouse cytotoxic T cells. The IL-15 was prepared at 100 μg/mL in sterile PBS buffer and added into a cell culture medium with desired concentrations. The measurement used colorimetric MTT assay to measure the number of CTLL-2 cells, reflecting the tendency of cell proliferation. The freshly prepared IL-15 G6A was used in this measurement and the ED₅₀ (50% effective dosage) of IL-15 G5A was 0.006 nM.

Table 6 showed the ED₅₀ values kept the same range (0.006-0.011 nM) for the IL-15 samples stored within 2 weeks. The cyclized IL-15 showed prolonged activity even after the storage period of 2 weeks.

TABLE 6
The ED₅₀ of the cyclized IL-15 G6A

| IL-15 G6A | ED₅₀ (nM) | R² |
|---|---|---|
| Fresh prepared | 0.006 | 0.92 |
| After 3 days | 0.007 | 0.98 |
| After 5 days | 0.007 | 0.97 |
| After 1 week | 0.009 | 0.98 |
| After 2 weeks | 0.011 | 0.97 |

### Example 6: The Invention of Cyclized IL-6 with Different Split Sites

Cyclized IL-6 invention was to prove the feasibility of using different split sites to design the cyclized interleukin. The resulted cyclized IL-6s contained the same sequence and structure.

The native amino acid sequence of IL-6 was shown in SEQ ID NO: 28. The intein chosen for preparing cyclized IL-6 was Npu DnaE intein.

Two sets of split IL-6 sequences were designed in the present invention. The first case was with the split site introduced at the junction between residues G54 and C55 (sequence number referencing to SEQ ID NO: 28) to produce a C-terminal fragment (C-target, SEQ ID NO: 29) and a N-terminal fragment (N-target, SEQ ID NO: 30), where the resides of C55 contained the side-chain nucleophile (—SH), responsible for cyclization reaction. The second case was with the split site introduced at the junction between residues T64 and C65 (sequence number referencing to SEQ ID NO: 28) to produce a C-terminal fragment (C-target, SEQ ID NO: 31) and a N-terminal fragment (N-target, SEQ ID NO: 32), where the resides C65 contained the side-chain nucleophile (—SH), responsible for cyclization reaction.

The present invention introduced a linker of GSGSG into the two activatable sequences of IL-6. The new arrangements of IL-6 in the present invention were shown in SEQ ID NOs: 33 to 34.

The two activatable cyclized IL-6 in the present invention comprised the amino acid sequences as shown in SEQ ID NO: 35 (IL-6 G5A) and SEQ ID NO: 36 (IL-6 G5B). The sequences of IL-6 used in the present invention were shown in Table 7.

TABLE 7
Sequence used in the present invention
for preparing cyclized IL-6s

Native IL-6 sequence
SEQ ID NO: 28
LTSSERIDKQIRYILDGISALRKETCNKSNMCESSKEALAENNLNLPKMA
EKDGCFQSGFNEETCLVKIITGLLEFEVYLEYLQNRFESSEEQARAVQMS
TKVLIQFLQKKAKNLDAITTPDPTTNASLLTKLQAQNQWLQDMTTHLILR
SFKEFLQSSLRALRQM
C-terminal fragment of IL-6 G5A sequence
SEQ ID NO: 29
CFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKE
FLQSSLRALRQM
N-terminal fragment of IL-6 G5A sequence
SEQ ID NO: 30
LTSSERIDKQIRYILDGISALRKETCNKSNMCESSKEALAENNLNLPKMA
EKDG
C-terminal fragment of IL-6 G5B sequence
SEQ ID NO: 31
CLVKIITGLLEFEVYLEYLQNRFESSEEQARAVQMSTKVLIQFLQKKAKN
LDAITTPDPTTNASLLTKLQAQNQWLQDMTTHLILRSFKEFLQSSLRALR
QM
N-terminal fragment of IL-6 G5B sequence
SEQ ID NO: 32
LTSSERIDKQIRYILDGISALRKETCNKSNMCESSKEALAENNLNLPKMA
EKDGCFQSGFNEET
The activatable polypeptide of IL-6 G5A
of the present invention
SEQ ID NO: 33
MIKIATRKYLGKQNVYDIGVERDHNFALKNGFIASNCFQSGFNEETCLVK
IITGLLEFEVYLEYLQNRFESSEEQARAVOMSTKVLIQFLQKKAKNLDAI
TTPDPTTNASLLTKLQAQNQWLQDMTTHLILRSFKEFLQSSLRALRQMGS
GSGLTSSERIDKQIRYILDGISALRKETCNKSNMCESSKEALAENNLNLP
KMAEKDGCLSYETEILTVEYGLLPIGKIVEKRIECTVYSVDNNGNIYTQP
VAQWHDRGEQEVFEYCLEDGSLIRATKDHKFMTVDGQMLPIDEIFERELD
LMRVDNLPNLEHHHHHH
The activatable polypeptide of IL-6 G5B
of the present invention
SEQ ID NO: 34
MIKIATRKYLGKQNVYDIGVERDHNFALKNGFIASNCLVKIITGLLEFEV
YLEYLQNRFESSEEQARAVQMSTKVLIQFLQKKAKNLDAITTPDPTTNAS
LLTKLQAQNQWLQDMTTHLILRSFKEFLQSSLRALRQMGSGSGLTSSERI
DKQIRYILDGISALRKETCNKSNMCESSKEALAENNLNLPKMAEKDGCFQ
SGFNEETCLSYETEILTVEYGLLPIGKIVEKRIECTVYSVDNNGNIYTQP
VAQWHDRGEQEVFEYCLEDGSLIRATKDHKFMTVDGQMLPIDEIFERELD
LMRVDNLPNLEHHHHHH
The cyclized IL-6 G5A from SEQ ID NO: 33
SEQ ID NO: 35
CFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKE
FLQSSLRALRQMGSGSGLTSSERIDKQIRYILDGISALRKETCNKSNMCE
SSKEALAENNLNLPKMAEKDG
The cyclized IL-6 G5B from SEQ ID NO: 34
SEQ ID NO: 36
CLVKIITGLLEFEVYLEYLQNRFESSEEQARAVQMSTKVLIQFLQKKAKN
LDAITTPDPTTNASLLTKLQAQNQWLQDMTTHLILRSFKEFLQSSLRALR
QMGSGSGLTSSERIDKQIRYILDGISALRKETCNKSNMCESSKEALAENN
LNLPKMAEKDGCFQSGFNEET

In Table 7, the sequences with underlines were designed by the present invention for cyclized IL-6s.

The two sets of split IL-6 sequences resulted in the same cyclized IL-6 sequences although the introduced split sites were different.

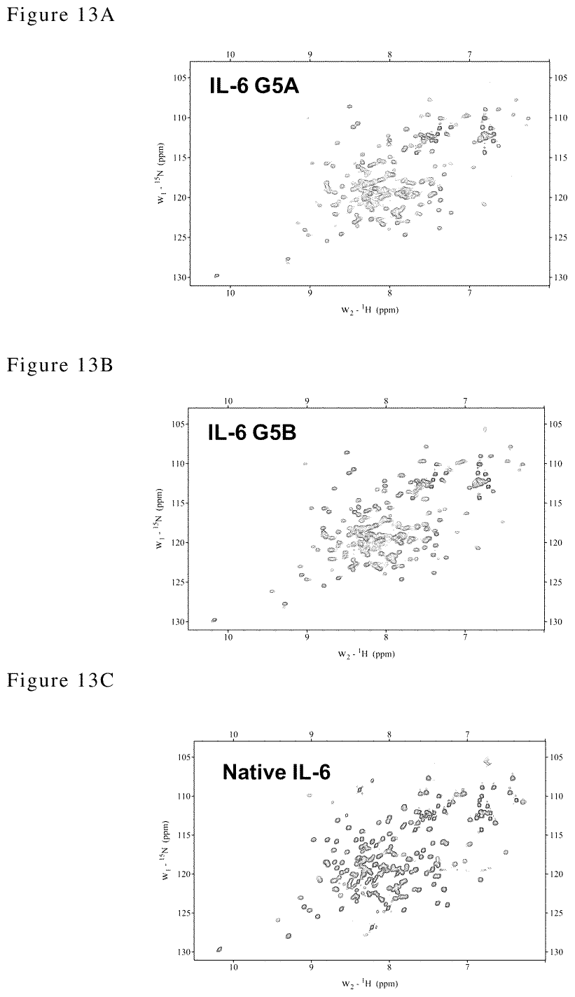

FIG. 13 showed the HSQC spectra of cyclized IL-6 G5A (FIG. 13A) and IL-6 G5B (FIG. 13B), and the native IL-6 (FIG. 13C). IL-6 G5A and IL-6 G5B showed the same spectra. The dispersive resonances indicated a well-folded structure. The HSQC spectra of the native IL-6 was a control. The spectral similarity proved the structural similarity between cyclized IL-6s and native IL-6. The NMR condition was with a buffer of 20 mM sodium phosphate, pH 6.0, 150 mM NaCl and temperature at 298K.

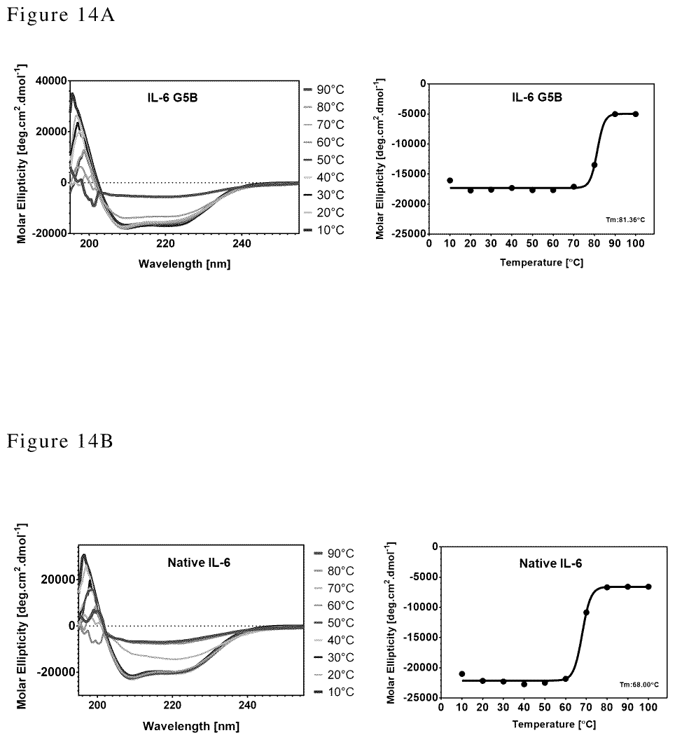

FIG. 14A showed the structural stability of cyclized IL-6 G5B, monitored by circular dichroism (CD) spectra at different temperatures. The thermal denaturation was monitored by CD spectra at one single wavelength of 208 nm. The measured melting temperature is 81° C. Cyclized IL-6 G5A showed the same denaturation profile of melting temperature of 82° C.

FIG. 14B showed the structural stability of native IL-6, monitored by circular dichroism (CD) spectra at different temperatures. The thermal denaturation was monitored by CD spectra at one single wavelength of 208 nm. The measured melting temperature was 68° C. The melting temperature of cyclized IL-6 was shown in Table 8.

TABLE 8
The melting temperature of cyclized IL-6

| | | Melting | | |
|---|---|---|---|---|
| | | temperature | | |
| | Melting | of native | | |
| | temperature | protein | ED₅₀ | The reported ED₅₀ |
| IL-6 G5A | 82° C. | 68° C. | 0.14 nM | 0.01-0.05 nM |
| IL-6 G5B | 81° C. | | 0.18 nM | |

### Example 7: The Invention of Cyclized Interleukins with Different Split Sites

In example 6, the invention of cyclized IL-6 could adopt different split sites. Therefore, with the same strategy, the present invention could extend to use other split sites to prepare cyclized IL-6. For example, in IL-6 case, the preferred split sites could be alternatively selected at the site between T25 and C26 or at the site between M31 and C32 where the later Cys resides contained the side-chain nucleophile.

Meanwhile, the invention of cyclized IL-2 and IL-15 could be alternatively designed by adopting different split sites. In IL-2 case, the preferred split sites could be alternatively selected at the site between M104 and C105 or at the site between F124 and C125. Meanwhile, In IL-15 case, the preferred split sites could be alternatively selected at the sites between S34 and C35 or at the site between G84 and C85 or at the site between E87 and C88.

Those skilled in the art recognize the foregoing outline as a description of the method for communicating hosted application information. The skilled artisan will recognize that these are illustrative only and that many equivalents are possible.

What is claimed is:

1. An activatable polypeptide which comprises, in the N-terminal to C-terminal direction: (1) a C-intein; (2) a C-interleukin; (3) a linker; (4) a N-interleukin and (5) a N-intein, wherein the C-interleukin and the N-interleukin are obtained by splitting a target interleukin, the splitting site is a site of an amino acid with a side-chain nucleophile in the peptide sequence of the target interleukin, and the N-terminus of the C-interleukin contains the amino acid with the side-chain nucleophile.

2. The activatable polypeptide of claim 1, wherein the target protein comprises interleukin-2, interleukin-15 or interleukin-6.

3. The activatable polypeptide of claim 1, wherein the target interleukin comprises the peptide sequence of SEQ ID No: 3, 4, 19 or 28.

4. The activatable polypeptide of claim 1, wherein the C-interleukin comprises the peptide sequence of SEQ ID NO: 5, and the N-interleukin comprises the peptide sequence of SEQ ID NO: 6.

5. The activatable polypeptide of claim 1, wherein the C-interleukin comprises the peptide sequence of SEQ ID NO: 20, and the N-interleukin comprises the peptide sequence of SEQ ID NO: 21.

6. The activatable polypeptide of claim 1, wherein the C-interleukin comprises the peptide sequence of SEQ ID NO: 29 or 31, and the N-interleukin comprises the peptide sequence of SEQ ID NO: 30 or 32.

7. The activatable polypeptide of claim 1, wherein the amino acid with the side-chain nucleophile comprises cysteine, serine or threonine.

8. The activatable polypeptide of claim 1, wherein the peptide sequence of the linker comprises GSGSGS, GSGSG, GGGGG, GSGS, or GSG.

9. The activatable polypeptide of claim 1, which comprises the peptide sequence of SEQ ID NO: 11 to 14, 22 to 24, 33 or 34.

10. A cyclized polypeptide which is in a circular form comprising the peptide sequence of SEQ ID NO: 9, 10, 15 to 18, 25 to 27, 35 or 36.


SEQUENCE LISTING (36 sequences)

SEQ ID NO: 1 - protein, 36 aa, synthetic construct
MIKIATRKYLGKQNVYDIGVERDHNFALKNGFIASN

SEQ ID NO: 2 - protein, 110 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 3 - protein, 133 aa, Homo sapiens [sequence omitted]

SEQ ID NO: 4 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 76 aa, synthetic construct
CLEEELKPLEEVLNLAQSKNFHLRPRDLISNINVIVLELKGSETTFMCEYADETATIVEFLNRWITFCQSIISTLT

SEQ ID NO: 6 - protein, 57 aa, synthetic construct
APTSSSTKKTQLQLEHLLLDLQMILNGINNYKNPKLTRMLTFKFYMPKKATELKHLQ

SEQ ID NO: 7 - protein, 284 aa, synthetic construct
MIKIATRKYLGKQNVYDIGVERDHNFALKNGFIASNCFNAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 8 - protein, 284 aa, synthetic construct
MIKIATRKYLGKQNVYDIGVERDHNFALKNGFIASNCFNAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 9 - protein, 138 aa, synthetic construct
CFNAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGS

SEQ ID NO: 10 - protein, 138 aa, synthetic construct
CFNAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGS

SEQ ID NO: 11 - protein, 284 aa, synthetic construct
MIKIATRKYLGKQNVYDIGVERDHNFALKNGFIASNCLEEELKPLEEVLNLAQSKNFHLRPRDLISNINVIVLELKGSETTFMCEYADETATIVEFLNRWITFCQSIISTLTGSGSGAPTSSSTKKTQLQLEHLLLDLQMILNGINNYKNPKLTRMLTFKFYMPKKATELKHLQCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 12 - protein, 284 aa, synthetic construct
MIKIATRKYLGKQNVYDIGVERDHNFALKNGFIASNCLEEELKPLEEVLNLAQSKNFHLRPRDLISNINVIVLELKGSETTFMCEYADETATIVEFLNRWITFCQSIISTLTGGGGGAPTSSSTKKTQLQLEHLLLDLQMILNGINNYKNPKLTRMLTFKFYMPKKATELKHLQCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 13 - protein, 283 aa, synthetic construct
MIKIATRKYLGKQNVYDIGVERDHNFALKNGFIASNCLEEELKPLEEVLNLAQSKNFHLRPRDLISNINVIVLELKGSETTFMCEYADETATIVEFLNRWITFCQSIISTLTGSGSAPTSSSTKKTQLQLEHLLLDLQMILNGINNYKNPKLTRMLTFKFYMPKKATELKHLQCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 14 - protein, 282 aa, synthetic construct
MIKIATRKYLGKQNVYDIGVERDHNFALKNGFIASNCLEEELKPLEEVLNLAQSKNFHLRPRDLISNINVIVLELKGSETTFMCEYADETATIVEFLNRWITFCQSIISTLTGSGAPTSSSTKKTQLQLEHLLLDLQMILNGINNYKNPKLTRMLTFKFYMPKKATELKHLQCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 15 - protein, 138 aa, synthetic construct
CLEEELKPLEEVLNLAQSKNFHLRPRDLISNINVIVLELKGSETTFMCEYADETATIVEFLNRWITFCQSIISTLTGSGSGAPTSSSTKKTQLQLEHLLLDLQMILNGINNYKNPKLTRMLTFKFYMPKKATELKHLQ

SEQ ID NO: 16 - protein, 138 aa, synthetic construct
CLEEELKPLEEVLNLAQSKNFHLRPRDLISNINVIVLELKGSETTFMCEYADETATIVEFLNRWITFCQSIISTLTGGGGGAPTSSSTKKTQLQLEHLLLDLQMILNGINNYKNPKLTRMLTFKFYMPKKATELKHLQ

SEQ ID NO: 17 - protein, 137 aa, synthetic construct
CLEEELKPLEEVLNLAQSKNFHLRPRDLISNINVIVLELKGSETTFMCEYADETATIVEFLNRWITFCQSIISTLTGSGSAPTSSSTKKTQLQLEHLLLDLQMILNGINNYKNPKLTRMLTFKFYMPKKATELKHLQ

SEQ ID NO: 18 - protein, 136 aa, synthetic construct
CLEEELKPLEEVLNLAQSKNFHLRPRDLISNINVIVLELKGSETTFMCEYADETATIVEFLNRWITFCQSIISTLTGSGAPTSSSTKKTQLQLEHLLLDLQMILNGINNYKNPKLTRMLTFKFYMPKKATELKHLQ

SEQ ID NO: 19 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 20 - protein, 73 aa, synthetic construct
CFLLELQVISLESGDASIHDTVENLIILANNSLSSNGNVTESGCKECEELEEKNIKEFLQSFVHIVQMFINTS

SEQ ID NO: 21 - protein, 41 aa, synthetic construct
NWVNVISDLKKIEDLIQSMHIDATLYTESDVHPSCKVTAMK

SEQ ID NO: 22 - protein, 266 aa, synthetic construct
MIKIATRKYLGKQNVYDIGVERDHNFALKNGFIASNCFLLELQVISLESGDASIHDTVENLIILANNSLSSNGNVTESGCKECEELEEKNIKEFLQSFVHIVQMFINTSGSGSGSNWVNVISDLKKIEDLIQSMHIDATLYTESDVHPSCKVTAMKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 23 - protein, 265 aa, synthetic construct
MIKIATRKYLGKQNVYDIGVERDHNFALKNGFIASNCFLLELQVISLESGDASIHDTVENLIILANNSLSSNGNVTESGCKECEELEEKNIKEFLQSFVHIVQMFINTSGSGSGNWVNVISDLKKIEDLIQSMHIDATLYTESDVHPSCKVTAMKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 24 - protein, 264 aa, synthetic construct
MIKIATRKYLGKQNVYDIGVERDHNFALKNGFIASNCFLLELQVISLESGDASIHDTVENLIILANNSLSSNGNVTESGCKECEELEEKNIKEFLQSFVHIVQMFINTSGSGSNWVNVISDLKKIEDLIQSMHIDATLYTESDVHPSCKVTAMKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 25 - protein, 120 aa, synthetic construct
CFLLELQVISLESGDASIHDTVENLIILANNSLSSNGNVTESGCKECEELEEKNIKEFLQSFVHIVQMFINTSGSGSGSNWVNVISDLKKIEDLIQSMHIDATLYTESDVHPSCKVTAMK

SEQ ID NO: 26 - protein, 119 aa, synthetic construct
CFLLELQVISLESGDASIHDTVENLIILANNSLSSNGNVTESGCKECEELEEKNIKEFLQSFVHIVQMFINTSGSGSGNWVNVISDLKKIEDLIQSMHIDATLYTESDVHPSCKVTAMK

SEQ ID NO: 27 - protein, 118 aa, synthetic construct
CFLLELQVISLESGDASIHDTVENLIILANNSLSSNGNVTESGCKECEELEEKNIKEFLQSFVHIVQMFINTSGSGSNWVNVISDLKKIEDLIQSMHIDATLYTESDVHPSCKVTAMK

SEQ ID NO: 28 - protein, 166 aa, Homo sapiens
LTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 29 - protein, 112 aa, synthetic construct
CFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 30 - protein, 54 aa, synthetic construct
LTSSERIDKQIRYILDGISALRKETCNKSNMCESSKEALAENNLNLPKMAEKDG

SEQ ID NO: 31 - protein, 102 aa, synthetic construct
CLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 32 - protein, 64 aa, synthetic construct
LTSSERIDKQIRYILDGISALRKETCNKSNMCESSKEALAENNLNLPKMAEKDGCFQSGFNEET

SEQ ID NO: 33 - protein, 317 aa, synthetic construct
MIKIATRKYLGKQNVYDIGVERDHNFALKNGFIASNCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMGSGSGLTSSERIDKQIRYILDGISALRKETCNKSNMCESSKEALAENNLNLPKMAEKDGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 34 - protein, 317 aa, synthetic construct
MIKIATRKYLGKQNVYDIGVERDHNFALKNGFIASNCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMGSGSGLTSSERIDKQIRYILDGISALRKETCNKSNMCESSKEALAENNLNLPKMAEKDGCFQSGFNEETCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEHHHHHH

SEQ ID NO: 35 - protein, 171 aa, synthetic construct
CFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMGSGSGLTSSERIDKQIRYILDGISALRKETCNKSNMCESSKEALAENNLNLPKMAEKDG

SEQ ID NO: 36 - protein, 171 aa, synthetic construct
CLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMGSGSGLTSSERIDKQIRYILDGISALRKETCNKSNMCESSKEALAENNLNLPKMAEKDGCFQSGFNEET